(19) United States
(12) Patent Application Publication
(10) Pub. No.: US 2024/0002908 A1
(43) Pub. Date: Jan. 4, 2024

(54) METHODS OF DETERMINING SURFACE GLYCAN DENSITY

(71) Applicant: The General Hospital Corporation, Boston, MA (US)

(72) Inventors: James T. Kurnick, Quincy, MA (US); Ian Seymour Dunn, Beverly, MA (US); Matthew M. Lawler, Madison, WI (US)

(21) Appl. No.: 18/036,008

(22) PCT Filed: Nov. 5, 2021
(86) PCT No.: PCT/US2021/058318

§ 371 Date: May 9, 2023

Related U.S. Application Data
(60) Provisional application No. 63/111,443, filed on Nov. 9, 2020.

(51) Int. Cl. C12Q 1/6816

(52) U.S. Cl. CPC C12Q 1/6816; C12Q 2600/166

(57) ABSTRACT

The present disclosure provides methods of determining surface glycan density of a cell.

### CROSS-REFERENCE TO RELATED APPLICATIONS

This application claims priority to U.S. Provisional Patent Application No. 63/111,443, filed Nov. 9, 2020. The contents of this application are incorporated herein by reference in its entirety.

### FIELD OF THE INVENTION

The present disclosure is directed, in part, to methods of determining surface glycan density of a cell.

### BACKGROUND

Sialic acid and other sugar-based metabolic pathways are ubiquitous in mammalian cellular and molecular physiologies. Placement of azides or other unnatural compounds on cell surfaces can be used, for example, in diagnostic and therapeutic processes. For example, where the density of surface glycans (which can display modified azide-bearing sugars) is abnormally high, a differential between normal and aberrant conditions can thereby be revealed. High surface glycan density in tumor cells in turn results in corresponding increased density of metabolically-placed surface azides or other artificial moieties of interest. The latter unnatural surface groups can act as molecular determinants for phenomena that inherently rely on molecular proximity, such as template-directed assembly of haplomers (i.e., (TAPER technology) (see, PCT Publications WO 14/197547, WO 16/089958, and WO 18/094195, and EP 3004351). On cells where surface azides are displayed, templates or haplomers may be immobilized on cell surfaces if they are modified with moieties that are bio-orthogonally reactive with azides, such as DBCO, to construct various therapeutic agents. Increased surface density of glycans can be a feature in distinguishing cancer cells from their normal counterparts, as well as for implementing TAPER-based specific assembly of desired products for ultimately killing the pathological cells. Thus, surface density of glycans can be useful information in both diagnostic and therapeutic processes. Accordingly, methods of assessing the relative surface densities of glycans is needed.

### SUMMARY

The present disclosure provides methods of determining surface glycan density of a cell, the method comprising the steps: a) contacting the cell with an azide-modified sugar; b) contacting the cell with a nucleic acid template comprising a 5′- or 3′-azide reactive molecule that is chemically reactable with an azide group, wherein the nucleic acid template has a first haplomer hybridization region and a second haplomer hybridization region; c) contacting the cell with a first haplomer comprising: i) a first oligonucleotide conjugated to a first ligand, wherein the first oligonucleotide is complementary to the first haplomer hybridization region; and ii) a first fragment of a reporter molecule conjugated to a first ligand binding domain; wherein the first ligand and first ligand binding domain associate with one another; d) contacting the cell with a second haplomer comprising: i) a second oligonucleotide conjugated to a second ligand, wherein the second oligonucleotide is complementary to the second haplomer hybridization region; and ii) a second fragment of the reporter molecule conjugated to a second ligand binding domain; wherein the second ligand and the second ligand binding domain associate with one another; whereby, upon hybridization of the first oligonucleotide to the first haplomer hybridization region and the second oligonucleotide to the second haplomer hybridization region, and refolding of the first fragment of the reporter molecule with the second fragment of the reporter molecule, a functional reporter molecule is produced; and e) detecting the amount of reporter molecule activity.

The present disclosure also provides methods of determining surface glycan density of a cell, the method comprising the steps: a) contacting the cell with an azide-modified sugar; b) contacting the cell with a first nucleic acid template comprising a 5′-azide reactive molecule that is chemically reactable with an azide group, wherein the nucleic acid template has a first haplomer hybridization region; c) contacting the cell with a second nucleic acid template comprising a 3′-azide reactive molecule that is chemically reactable with an azide group, wherein the nucleic acid template has a second haplomer hybridization region; d) contacting the cell with a first haplomer comprising: i) a first oligonucleotide conjugated to a first ligand, wherein the first oligonucleotide is complementary to the first haplomer hybridization region; and ii) a first fragment of a reporter molecule conjugated to a first ligand binding domain; wherein the first ligand and the first ligand binding domain associate with one another; e) contacting the cell with a second haplomer comprising: i) a second oligonucleotide conjugated to a second ligand, wherein the second oligonucleotide is complementary to the second haplomer hybridization region; and ii) a second fragment of the reporter molecule conjugated to a second ligand binding domain; wherein the second ligand and the second ligand binding domain associate with one another; whereby, upon hybridization of the first oligonucleotide to the first haplomer hybridization region and the second oligonucleotide to the second haplomer hybridization region, and refolding of the first fragment of the reporter molecule with the second fragment of the reporter molecule, a functional reporter molecule is produced; and f) detecting the amount of reporter molecule activity.

The present disclosure also provides methods of determining surface glycan density of a cell, the method comprising the steps: a) contacting the cell with an azide-modified sugar; b) contacting the cell with a bifunctional compound, wherein the bifunctional compound comprises an azide reactive molecule that is chemically reactable with an azide group, and a small molecule ligand that binds to an FKBP domain or FRB domain, wherein the bifunctional compound associates with a first glycan molecule, and another bifunctional compound associates with a second glycan molecule; c) contacting the cell with a first haplomer comprising an FKBP domain or FRB domain conjugated to a first fragment of a reporter molecule; d) contacting the cell with a second haplomer comprising an FKBP domain or FRB domain conjugated to a second fragment of the reporter molecule; whereby, upon association between the FKBP domain or FRB domain of the first haplomer and the small molecule ligand of the bifunctional molecule of a first glycan molecule and the association between the FKBP domain or FRB domain of the second haplomer and the small molecule ligand of the bifunctional molecule of a second glycan molecule in sufficient proximity to the first glycan molecule, and refolding of the first fragment of the reporter molecule with the second fragment of the reporter molecule, a functional reporter molecule is produced; and e) detecting the amount of reporter molecule activity.

### BRIEF DESCRIPTION OF THE DRAWINGS

FIG. 1 shows a representation of the principles of cis/trans Gaussia complementation for cell surface glycan concentration, involving masking haplomer hybridization sites on a template with complementary oligonucleotides.

FIG. 2 shows a representation of the principles of cis/trans Gaussia complementation for cell surface glycan concentration, involving Gaussia haplomers constructed with MFL2-modified oligonucleotides complementary to a template.

FIG. 3 shows a representation of the principles of cis/trans Gaussia complementation for cell surface glycan concentration, involving Gaussia cis-SP complementation.

FIG. 4 shows a representation of the principles of cis/trans Gaussia complementation for cell surface glycan concentration, involving Gaussia trans-SP complementation under conditions of high cell surface glycan density.

FIG. 5 shows a representation of the principles of half-template trans-complementation assay, in which DBCO-half-templates are located to cell surfaces labeled metabolically with azide.

FIG. 6 shows a representation of the principles of half-template trans-complementation assay, involving Gaussia reporter assembly by trans-complementation.

FIG. 7 shows a representation of the principles of a surface density assay by surface placement of an FKBP-binding compound and subsequent haplomer assembly, involving MFL4-PD surface labeling of cells labeled with surface azide.

FIG. 8 shows a representation of the principles of a surface density assay by surface placement of an FKBP-binding compound and subsequent haplomer assembly, involving FKBP-labeled haplomer assembly for high-density placement of MFL4-PD on cell surfaces.

FIG. 9 shows a representation of the principles of density-driven split protein assembly by surface azide reactions.

### DETAILED DESCRIPTION

Numerous techniques exist for assembly of a molecule on the surface of a cell or within a cell. For example, several processes for the templated assembly of molecules by proximity-enhanced reactivity have been described (see, PCT Publications WO 14/197547, WO 17/205277, WO 18/94070, WO 18/94195, WO 18/93978, and WO 19/032942). For example, novel structures can be assembled on cellular nucleic acid templates which define pathogenic or otherwise undesirable cell classes. Such templated assembly processes can be used to target the cell types of interest for destruction. Pairs of modified oligonucleotides carrying specially tailored and mutually reactive groups can assemble molecules with predetermined functions following co-annealing in spatial proximity on a target cellular template (i.e., Template Assembly by Proximity-Enhanced Reactivity (TAPER). Proteins can be assembled via folding or dimerization using nucleic acid molecule templates which can be used to combat pathogenic or otherwise undesirable cells or cell products. Such templated assembly processes can be used to target the cell types of interest for destruction. Pairs of modified oligonucleotides carrying specially tailored and mutually reactive ligands can assemble proteins with predetermined functions following templated assembly. Unlike other forms of protein complementation (such as the alpha-complementation of beta-galactosidase) where pre-folded subunits interact, some of the methods described herein involve split-protein approaches characterized by the facilitation of mature folding pathways through enforced spatial proximity. Consequently, split-protein fragments in isolation cannot recapitulate the functional profile of their corresponding parental protein, and fragment background functional levels are accordingly extremely low.

In many of these processes, a template polynucleotide is delivered to the cell, such as to the surface of the cell, whereby the template polynucleotide can serve as a substrate by which to target the cell for the assembly of a molecule. The assembled molecule can then serve, for example, as a means to destroy the cell (such as by being a toxin) or by acting as a target for a therapeutic compound (such as by being an antigen for a therapeutic antibody). Numerous methods for the delivery of the template polynucleotide are set forth in the foregoing processes. A method of targeting such TAPER reagents to particular cells can involved targeting cells having azide-modified glycans on their surface. In reference thereto, methods of determining surface glycan density are disclosed herein.

The terminology used herein is for the purpose of describing particular embodiments only and is not intended to be limiting. The terms used in this disclosure adhere to standard definitions generally accepted by those having ordinary skill in the art. In case any further explanation might be needed, some terms have been further elucidated below.

As used herein, the singular forms “a,” “an,” and “the” include plural references unless the content clearly dictates otherwise.

As used herein, the phrase “functional reporter molecule” refers to a reporter molecule that has measurable function created by the association of two fragments of the reporter molecule via the assembly processes described herein, wherein each fragment of the reporter molecule by itself has no measurable function.

As used herein, the term “alkyl” means a saturated hydrocarbon group which is straight-chained or branched. In some embodiments, the alkyl group has from 1 to 20 carbon atoms, from 2 to 20 carbon atoms, from 2 to 16 carbon atoms, from 4 to 12 carbon atoms, from 4 to 16 carbon atoms, from 4 to 10 carbon atoms, from 1 to 10 carbon atoms, from 2 to 10 carbon atoms, from 1 to 8 carbon atoms, from 2 to 8 carbon atoms, from 1 to 6 carbon atoms, from 2 to 6 carbon atoms, from 1 to 4 carbon atoms, from 2 to 4 carbon atoms, from 1 to 3 carbon atoms, or 2 or 3 carbon atoms. Examples of alkyl groups include, but are not limited to, methyl (Me), ethyl (Et), propyl (e.g., n-propyl and isopropyl), butyl (e.g., n-butyl, t-butyl, isobutyl), pentyl (e.g., n-pentyl, isopentyl, neopentyl), hexyl, isohexyl, heptyl, octyl, nonyl, 4,4 dimethylpentyl, decyl, undecyl, dodecyl, 2,2,4-trimethylpentyl, 2-methyl-1-propyl, 2-methyl-2-propyl, 2-methyl-1-butyl, 3-methyl-1-butyl, 2-methyl-3-butyl, 2-methyl-1-pentyl, 2,2-dimethyl-1-propyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, and the like.

As used herein, the term “base” refers to a molecule containing a purine or pyrimidine group, or an artificial analogue, that forms a binding pair with another corresponding base via Watson-Crick or Hoogsteen bonding interactions. Bases further contain groups that facilitate covalently joining multiple bases together in a polymer, such as an oligomer. Non-limiting examples include nucleotides, nucleosides, peptide nucleic acid residues, or morpholino residues.

As used herein, the terms “bind,” “binds,” “binding,” and “bound” refer to a stable interaction between two molecules that are close to one another. The terms include physical interactions, such as chemical bonds (either directly linked or through intermediate structures), as well as non-physical interactions and attractive forces, such as electrostatic attraction, hydrogen bonding, and van der Waals/dispersion forces.

As used herein, the phrase “chemical linker” refers to a molecule that binds one haplomer to another haplomer or one moiety to another moiety on different compounds. A linker may be comprised of branched or unbranched covalently bonded molecular chains.

As used herein, the terms “comprising” (and any form of comprising, such as “comprise”, “comprises”, and “comprised”) and “having” (and any form of having, such as “have” and “has”) are inclusive and open-ended and include the options following the terms, and do not exclude additional, unrecited elements, or method steps.

As used herein, the term “contacting” means bringing together a compound and a cell, or a compound with another compound in an in vitro system or an in vivo system.

As used herein, the phrase “ethylene glycol unit” means a polymer of —(O—CH₂—CH₂)ₙ—O—, wherein n is from 1 to about 20. A polyethylene glycol (PEG) having 4 ethylene glycol units (i.e., —(O—CH₂—CH₂)₄—O—) is referred to herein as PEG4.

As used herein, the term “haplomer” refers to a compound comprising: i) an oligonucleotide conjugated to a ligand, wherein the oligonucleotide is complementary to a haplomer hybridization region; and ii) a fragment of a reporter molecule conjugated to a ligand binding domain, wherein the ligand and ligand binding domain associate with one another. The haplomer can also be a compound comprising an FKBP domain or FRB domain conjugated to a fragment of a reporter molecule.

As used herein, the terms “oligomer”, “oligo”, and “oligonucleotide” refer to a molecule comprised of multiple units where some or all of the units are bases capable of forming Watson-Crick or Hoogsteen base-pairing interactions, allowing sequence-specific binding to nucleic acid molecules in a duplex or multiplex structure. Non-limiting examples include, but are not limited to, oligonucleotides, peptide nucleic acid oligomers, and morpholino oligomers.

As used herein, the term “sample” refers to any system that haplomers can be administered into, where nucleic acid templated assembly may occur. Examples of samples include, but are not limited to, fixed or preserved cells, whole organisms, tissues, tumors, lysates, or in vitro assay systems.

As used herein, the phrase “selectively-reactive moiety” refers to a moiety that can react readily with a corresponding selectively-reactive moiety, but does not readily react with natural biomolecules. A modification of a sugar molecule (e.g., azide) and the counterpart molecule biding thereto (e.g., azide-reactive molecule) can be selectively-reactive moieties.

At various places herein, substituents of compounds may be disclosed in groups or in ranges. Designation of a range of values includes all integers within or defining the range (including the two endpoint values), and all subranges defined by integers within the range. It is specifically intended that the disclosure include each and every individual subcombination of the members of such groups and ranges. For example, the term “C₁₋₆alkyl” is specifically intended to individually disclose methyl, ethyl, propyl, C₄alkyl, C₅alkyl, and C₆alkyl.

It should be appreciated that particular features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

The structures depicted herein may omit necessary hydrogen atoms to complete the appropriate valency. Thus, in some instances a carbon atom or nitrogen atom may appear to have an open valency (i.e., a carbon atom with only two bonds showing would implicitly also be bonded to two hydrogen atoms; in addition, a nitrogen atom with a single bond depicted would implicitly also be bonded to two hydrogen atoms). For example, “—N” would be considered by one skilled in the art to be “—NH₂.” Thus, in any structure depicted herein wherein a valency is open, one or more hydrogen atoms, as appropriate, is implicit, and is only omitted for brevity.

The compounds described herein can also include all isotopes of atoms occurring in the intermediates or final compounds. Isotopes include those atoms having the same atomic number but different mass numbers. For example, isotopes of hydrogen include tritium and deuterium. Carbon (¹²C) can be replaced at any position with ¹³C or ¹⁴C. Nitrogen (¹⁴N) can be replaced with ¹⁵N. Oxygen (¹⁶O) can be replaced at any position with ¹⁷O or ¹⁸O. Sulfur (³²S) can be replaced with ³³S, ³⁴S or ³⁶S. Chlorine (³⁵Cl) can be replaced with ³⁷Cl. Bromine (⁷⁹Br) can be replaced with ⁸¹Br.

In some embodiments, the compounds, or salts thereof, are substantially isolated. Partial separation can include, for example, a composition enriched in any one or more of the compounds described herein. Substantial separation can include compositions containing at least about 50%, at least about 60%, at least about 70%, at least about 80%, at least about 90%, at least about 95%, at least about 97%, or at least about 99% by weight of any one or more of the compounds described herein, or salt thereof. Methods for isolating compounds and their salts are routine in the art.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

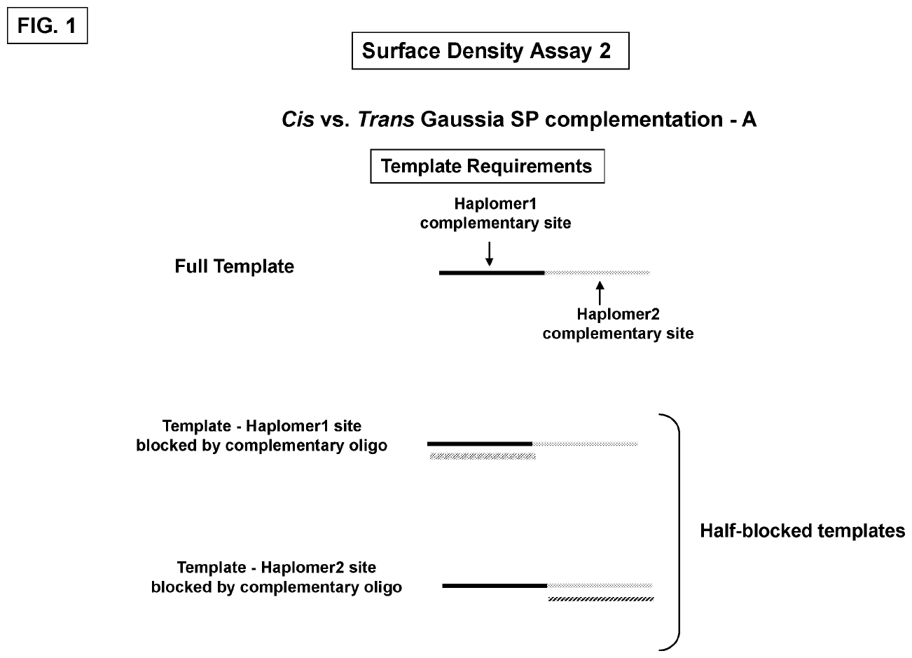

### A) Cis-Trans Gaussia Split-Protein Assembly (FIGS. 1-4)

A cis vs. trans Gaussia assay that can be used as a measurement strategy for surface glycan density. In such embodiments, a template is used either as normal with two sites for haplomer assembly accessible, or with each of the two sites separately blocked by hybridization with a complementary oligonucleotide (see, FIG. 1). In such a “masked” or half-blocked configuration, only haplomers complementary to the remaining open unhybridized site can anneal to the template (see, FIG. 1).

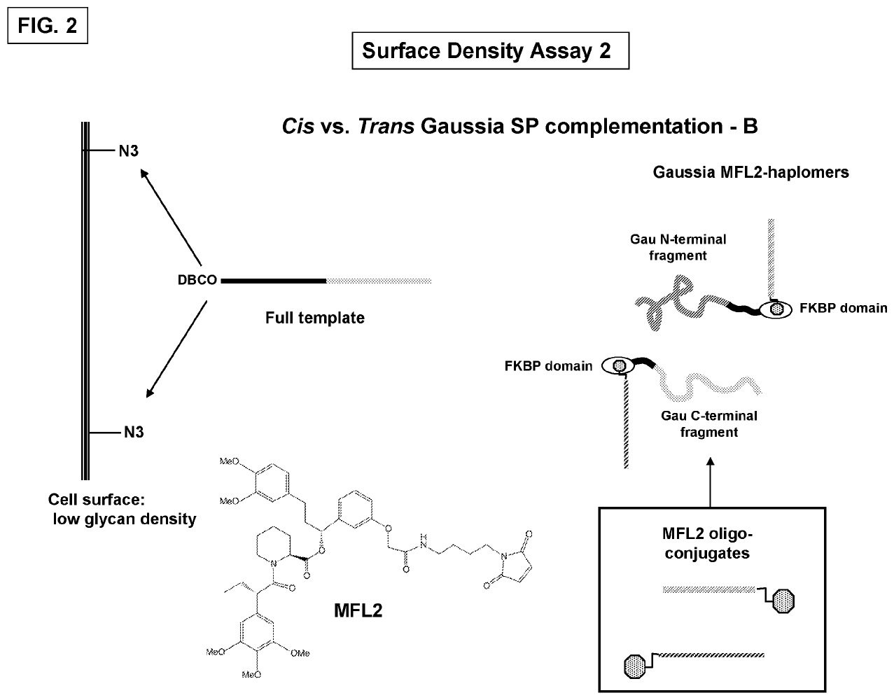

For a split-protein-TAPER (SP-TAPER; PCT Publication WO 16/089958) haplomer approach, Gaussia N-terminal and C-terminal haplomers are created using oligonucleotides complementary to the template of choice (see, FIG. 2). In some embodiments, this is achieved by means of a bifunctional compound (MFL2) which can be used to form conjugates with thiol-labeled oligonucleotides, and subsequently to specifically associate with modified versions of the FK506-binding protein domain (FKBP; PCT Publication WO 18/094195). By such means, proteins bearing FKBP domains can be efficiently tagged with desired oligonucleotides (see, FIG. 2).

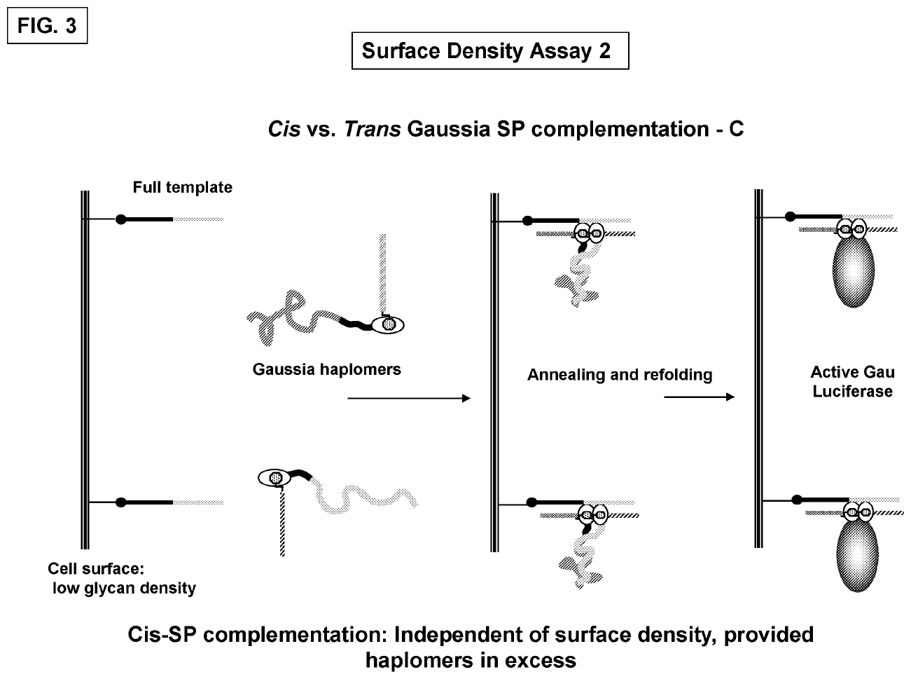

For normal cis-complementation, both Gaussia haplomers anneal on a common template, resulting in the proximal positioning of both N- and C-terminal protein fragments, and subsequent refolding and activity generation (assayable by luminescence; see, FIG. 3). In such circumstances, cis-complementation can occur irrespective of surface template density. The levels of supplied haplomers is preferably in excess. Should that not no longer be the case, the efficiency of SP reconstitution will fall as a consequence of the template titration effect. For in vitro assays, haplomer levels can be maintained such that they exceed even very high levels of surface labeling (where ≥10⁷ initial azide surface markers are present). In any case, this problem is remediable by the application of the Locked-TAPER system (PCT Publication WO 18/094070), where appropriate Locked-TAPER Gaussia haplomers are deployed.

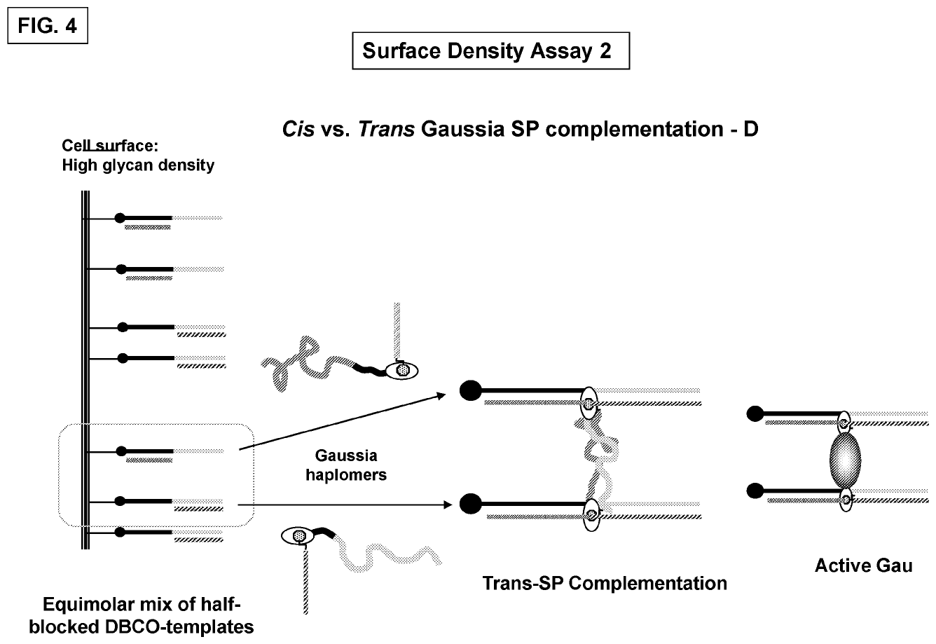

When an equimolar mix of DBCO-templates masked at either haplomer binding site is used for surface cell labeling, there is no possibility for normal cis-Gaussia SP-templating. Nevertheless, where the two Gaussia haplomers have separately templated on their masked templates in proximity to each other, it is possible for them to fold together as a trans-complementation reaction (see, FIG. 4).

Measurement of the generation of an assayable reporter response (as with luminescence for Gaussia) thus allows the assessment of surface density, where signal intensity of trans-complementation (with masked templates) is taken as proportional to the degree of cell surface glycan density. Concomitant measuring of reporter activity from the same labeled cells treated with unmasked template (cis-complementation) therefore allows a cis/trans activity ratio as the best measure of surface density.

The present disclosure provides methods of determining surface glycan density of a cell, the method comprising the steps: a) contacting the cell with an azide-modified sugar; b) contacting the cell with a nucleic acid template comprising a 5′- or 3′-azide reactive molecule that is chemically reactable with an azide group, wherein the nucleic acid template has a first haplomer hybridization region and a second haplomer hybridization region; c) contacting the cell with a first haplomer comprising: i) a first oligonucleotide conjugated to a first ligand, wherein the first oligonucleotide is complementary to the first haplomer hybridization region; and ii) a first fragment of a reporter molecule conjugated to a first ligand binding domain; wherein the first ligand and first ligand binding domain associate with one another; d) contacting the cell with a second haplomer comprising: i) a second oligonucleotide conjugated to a second ligand, wherein the second oligonucleotide is complementary to the second haplomer hybridization region; and ii) a second fragment of the reporter molecule conjugated to a second ligand binding domain; wherein the second ligand and the second ligand binding domain associate with one another; whereby, upon hybridization of the first oligonucleotide to the first haplomer hybridization region and the second oligonucleotide to the second haplomer hybridization region, and refolding of the first fragment of the reporter molecule with the second fragment of the reporter molecule, a functional reporter molecule is produced; and e) detecting the amount of reporter molecule activity.

In any of the embodiments described herein, the cell or cells are contacted with an azide-modified sugar. In any of the embodiments described herein, the cell can be any desired target cell. In some embodiments, the cell is a virus infected cell, a tumor cell, a cell infected with a microbe, or a cell that produces a molecule that leads to a disease, such as a cell that produces an antibody that induces allergy, anaphylaxis, or autoimmune disease, or a cytokine that mediates a disease. The cells can be cells of the immune system that are contributing to autoimmunity such as cells of the adaptive or innate immune systems, transplant rejection, or an allergic response. The cells described herein can be contacted with any of the azide-modified sugars described herein either in vitro or in vivo.

In any of the embodiments described herein, the azide-modified sugar is azido-N-acetylmannosamine (AzNAM), azido-N-acetylglucosamine (AzGlcNAc), azido-N-acetylgalactosamine (AGalNAc), or azido-N-acetylneuraminic acid (AzNANA). In some embodiments, the azide-modified sugar is AzNAM. In some embodiments, the azide-modified sugar is AzGlcNAc. In some embodiments, the azide-modified sugar AGalNAc. In some embodiments, the azide-modified sugar is AzNANA. In any of the embodiments described herein, the azide-modified sugar is acetylated at 1, 2, 3, or 4 positions. In some embodiments, the azide-modified sugar is acetylated at 1 position. In some embodiments, the azide-modified sugar is acetylated at 2 positions. In some embodiments, the azide-modified sugar is acetylated at 3 positions. In some embodiments, the azide-modified sugar is acetylated at 4 positions.

In any of the embodiments described herein, the cell or cells are contacted with a nucleic acid template comprising a 5′- or 3′-azide reactive molecule that is chemically reactable with an azide group. In any of the embodiments described herein, the azide reactive molecule is dibenzocyclooctyne (DBCO), bicyclo[6.1.0]nonyne (BCN), methyltetrazine, or trans-cyclooctene (TCO). In some embodiments, the azide reactive molecule is DBCO. In some embodiments, the azide reactive molecule is BCN. In some embodiments, the azide reactive molecule is methyltetrazine. In some embodiments, the azide reactive molecule is TCO. In addition, the nucleic acid template has a first haplomer hybridization region and a second haplomer hybridization region.

In some embodiments, the nucleic acid template comprising the 5′- or 3′-azide reactive molecule is a mixture of two nucleic acid templates comprising the 5′- or 3′-azide reactive molecule. The first nucleic acid template comprising the 5′- or 3′-azide reactive molecule is hybridized to a blocking oligonucleotide that is complementary to the first haplomer hybridization region. The second nucleic acid template comprising the 5′- or 3′-azide reactive molecule is hybridized to a blocking oligonucleotide that is complementary to the second haplomer hybridization region.

In any of the embodiments described herein, the cell or cells are contacted with a first haplomer comprising: i) a first oligonucleotide conjugated to a first ligand, wherein the first oligonucleotide is complementary to the first haplomer hybridization region of the nucleic acid template; and ii) a first fragment of a reporter molecule conjugated to a first ligand binding domain, wherein the first ligand and first ligand binding domain associate with one another.

In any of the embodiments described herein, the cell or cells are contacted with a second haplomer comprising: i) a second oligonucleotide conjugated to a second ligand, wherein the second oligonucleotide is complementary to the second haplomer hybridization region of the nucleic acid template; and ii) a second fragment of the reporter molecule conjugated to a second ligand binding domain, wherein the second ligand and the second ligand binding domain associate with one another. Upon hybridization of the first oligonucleotide to the first haplomer hybridization region and the second oligonucleotide to the second haplomer hybridization region, and refolding of the first fragment of the reporter molecule with the second fragment of the reporter molecule, a functional reporter molecule is produced.

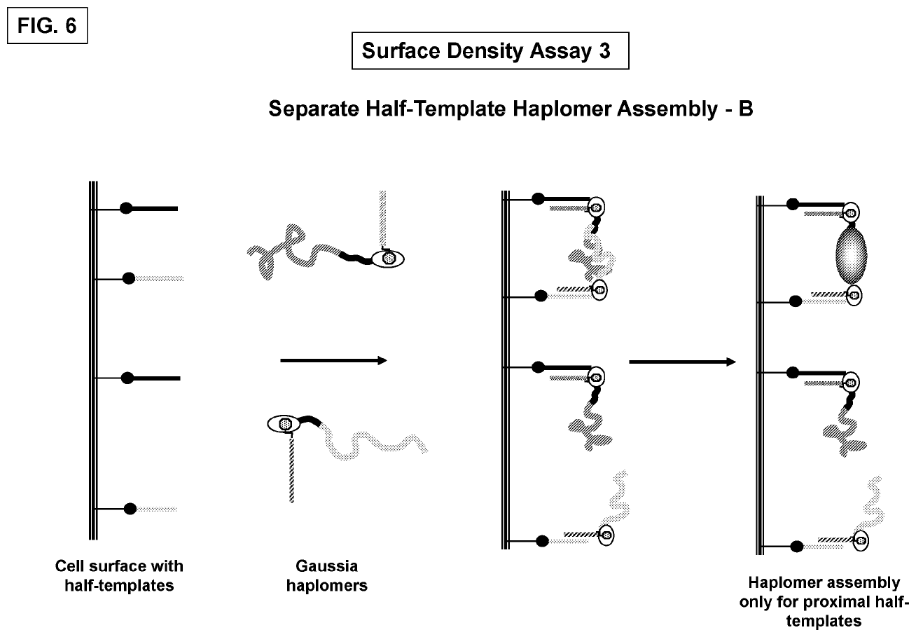

In some embodiments, the first ligand and second ligand are small molecule ligands. In some embodiments, the small molecule ligand is an FKBP-binding compound. In some embodiments, the FKBP-binding compound is chosen from FKM-NHS, FKM-sulfo-NHS, FKM-PEG₁₋₆-NHS, monovalent FKBP Ligand-2 (MFL2), FKM-PEG₁₋₆-MTZ-NHS, and FKM-PEG₁₋₆-TCO-NHS. In some embodiments, the FKBP-binding compound is FKM-NHS. In some embodiments, the FKBP-binding compound is FKM-sulfo-NHS. In some embodiments, the FKBP-binding compound is FKM-PEG₁₋₆-NHS. In some embodiments, the FKBP-binding compound is monovalent FKBP Ligand-2 (MFL2). In some embodiments, the FKBP-binding compound is FKM-PEG₁₋₆-MTZ-NHS. In some embodiments, the FKBP-binding compound is FKM-PEG₁₋₆-TCO-NHS. For example, some embodiments use modifications of the FKM-NHS series of compounds (see, FIGS. 6-8 of PCT Publication WO 18/094195) with selectively-reactive moiety side chains. In some embodiments, FKM-PEG₁₋₆-NHS is modified at the C2 position of the PEG chain with a methyltetrazine group (FKM-PEG₁₋₆-MTZ-NHS), or a trans-cyclooctene group (FKM-PEG₁₋₆-TCO-NHS)

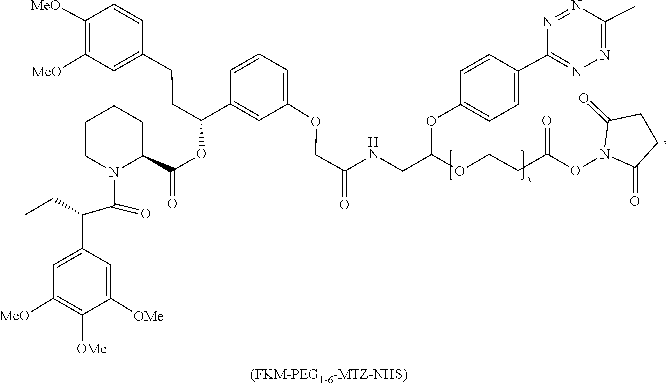

where
x is from 1 to 6; and FKM-PEG3-TCO-NHS is

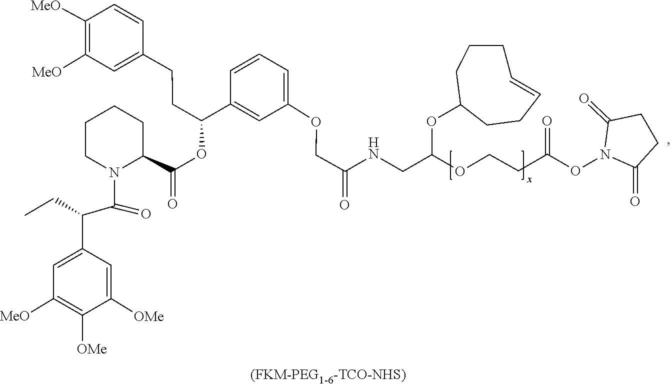

where x is from 1 to 6.

In some embodiments, when the first ligand and second ligand are small molecule ligands as set forth above, the first ligand binding domain and second ligand binding domains are FKBP domains or FRB domains. In some embodiments, the FKBP domain is a mutant FKBP domain. In some embodiments, the mutant FKBP domain is the F36V FKBP mutant domain comprising the amino acid sequence GVQVETISPGDGRTFPKRGQTCVVHYTGM LEDGKKVDSSRDRNKPFKFMLGKQEVIRGWEEGVAQMSVGQRAKLTISPDYAYGA TGHPGIIPPHATLVFDVELLKLE (SEQ ID NO:1) or MGVQVETISPGDGRTFPKRGQTC VVHYTGMLEDGKKVDSSRDRNKPFKFMLGKQEVIRGWEEGVAQMSVGQRAKLTIS PDYAYGATGHPGIIPPHATLVFDVELLKLE (SEQ ID NO:2). In some embodiments, the mutant FKBP domain comprises a C22S, C22A, or C22V substitution. In some embodiments, the FRB domain comprises a C61S, C61A, or C61V substitution.

In some embodiments, the first ligand and first ligand binding domain and/or the second ligand and second ligand binding domain are small interactive protein domain pairs. In some embodiments, the relatively small mutually interactive protein domains (e.g., fragments of proteins) are leucine zippers. Suitable examples of interactive protein domains are the c-jun and c-fos zipper domains, which generally are polypeptides of less than 50 amino acid residues, including helix-initiating and helix-terminating segments. While c-jun can form homodimers, c-fos cannot; and c-fos:c-jun heterodimers are significantly more stable than c-jun:c-jun homodimers. In some embodiments, the small interactive protein domain pairs are chosen from jun/fos, mad/max, myc/max, and NZ/CZ domains. In some embodiments, the small interactive protein domain pair is jun/fos (see, FIG. 14 of PCT Publication WO 18/094195). In some embodiments, the small interactive protein domain pair is mad/max. In some embodiments, the small interactive protein domain pair is myc/max. In some embodiments, the small interactive protein domain pair is NZ/CZ domains. In some embodiments, the interactive protein domain pair is antiparallel zippers, such as that from Thermus thermophilus seryl-tRNA synthetase.

In embodiments using small interactive protein domains as ligands, the polarity of the conjugation of the small domain tag should be taken into account. This can be exemplified with the particular embodiments using fos-jun heterodimerization, where the leucine zipper interaction occurs with a parallel orientation. If haplomers have appended c-Jun tags such that their c-Jun helices are in a parallel orientation following hybridization (see, FIG. 15 of PCT Publication WO 18/094195), then subsequent complex formation with c-fos will orient in a parallel sense; the reverse situation may disfavor dimerization between the protein segments of interest (see, FIG. 15 of PCT Publication WO 18/094195). However, for certain other applications, an antiparallel orientation can be beneficial. For this reason, it is advantageous if strategies exist for conjugating 5′ or 3′ polynucleotide ends with small protein tags by either their N- or C-termini.

In some embodiments, a sequence for making N-terminal conjugates of c-jun can be a 47-mer, where the N-terminal cysteine is shown, and the bold sequences denote helical boundaries: CSGGASLERIARLEEKVKTLKAQNSELASTANMLREQVAQLKQKGAP (SEQ ID NO:11). In some embodiments, a sequence for making C-terminal conjugates of c-jun can be a 49-mer, where the C-terminal cysteine is shown, and the bold sequences denote helical boundaries: SGASLERIARLEEKVKTLKAQNSELASTANMLREQVAQLKQKGA PSGGC (SEQ ID NO:12). In some embodiments, mutants of c-jun are used that cannot form homodimers, but which can still heterodimerize with c-fos. Such modified sequences with N-terminal cysteine residues include, but are not limited to: CSGGASLERIARLEEKVKSFKA QNSENASTANMLREQVAQLKQKGAP (SEQ ID NO:13), where bold residues denote changes from wild-type, and double-underlined sequences denote helical boundaries. Such modified sequences with C-terminal cysteine residues include, but are not limited to: SGAS LERIARLEEKVKSFKAQNSENASTANMLREQVAQLKQKGAPSGGC (SEQ ID NO:14), where bold residues denote changes from wild-type, and double-underlined sequences denote helical boundaries. Additional c-jun sequences include CSGASLERIARLEEKVKSFKAQNS ENASTANMLREQVAQLKQKGAP (SEQ ID NO:15) and GASLERIARLEEKVKTLKAQ NSELASTANMLREQVAQLKQKGAPSGGC (SEQ ID NO: 16).

In some embodiments, a sequence for making conjugates of c-fos can be a 41-mer, where the bold sequences denote helical boundaries: ASRELTDTLQAETDQLEDEKSALQ TEIANLLKEKEKLEGAP (SEQ ID NO:17). In some embodiments, the c-fos domain comprises the amino acid sequence ASRETDTLQAETDQLEDEKSALQTEIANLLKEKEK LEGAP (SEQ ID NO:18) or SGASRELTDTLQAETDQLEDEKSALQTEIANLLKEKEKLE GAP (SEQ ID NO:19). Additional extended serine-glycine linkers can be inserted between the c-fos sequence and the sequence to which it is linked.

In some embodiments, the NZ domain comprises the amino acid sequence ALKKEL QANKKELAQLKWELQALKKELAQ (SEQ ID NO:20), and the CZ domain comprises the amino acid sequence EQLEKKLQALEKKLAQLEWKNQALEKKLAQ (SEQ ID NO:21).

In any of the embodiments described herein, the reporter molecule is chosen from a luminescent protein, murine dihydrofolate reductase (DHFR), S. cerevisiae ubiquitin, β-lactamase, and Herpes simplex virus type 1 thymidine kinase. In some embodiments, the reporter molecule is a luminescent protein. In some embodiments, the reporter molecule is DHFR. In some embodiments, the reporter molecule is S. cerevisiae ubiquitin. In some embodiments, the reporter molecule is β-lactamase. In some embodiments, the reporter molecule is a Herpes simplex virus type 1 thymidine kinase. In any of the embodiments described herein, the reporter molecule can also be chosen from a choramphenicol acetyl transferase, a β-galactosidase, and a β-glucuronidase. In some embodiments, the reporter molecule is a choramphenicol acetyl transferase. In some embodiments, the reporter molecule is a β-galactosidase. In some embodiments, the reporter molecule is a 8-glucuronidase.

In some embodiments, when the reporter molecule is DHFR, one of the first fragment of the reporter molecule and the second fragment of the reporter molecule comprises amino acids 1-105 of DHFR, and the other of the first fragment of the reporter molecule and second fragment of the reporter molecule comprises amino acids 106-186 of DHFR.

In some embodiments, when the reporter molecule is S. cerevisiae ubiquitin, one of the first fragment of the reporter molecule and the second fragment of the reporter molecule comprises amino acids 1-34 (MQIFVKTLTGKTITLEVESSDTIDNVKSKIQDKE; SEQ ID NO:3), and the other of the first fragment of the reporter molecule and second fragment of the reporter molecule comprises amino acids 35-76 (GIPPDQQRLIFAGKQLEDGRTLSDY NIQKESTLHLVLRLRGG; SEQ ID NO:4).

In some embodiments, when the reporter molecule is β-lactamase, one of the first fragment of the reporter molecule and the second fragment of the reporter molecule comprises amino acids 25-197 of β-lactamase, and the other of the first fragment of the reporter molecule and second fragment of the reporter molecule comprises amino acids 198-286 of β-lactamase.

In some embodiments, when the reporter molecule is Herpes simplex virus type 1 thymidine kinase, one of the first fragment of the reporter molecule and the second fragment of the reporter molecule comprises amino acids 1-265 of Herpes simplex virus type 1 thymidine kinase, and the other of the first fragment of the reporter molecule and second fragment of the reporter molecule comprises amino acids 266-376 of Herpes simplex virus type 1 thymidine kinase.

In some embodiments, the reporter molecule is a luminescent protein including, but not limited to, Gaussia luciferase, superfolder GFP (sfGFP), Renilla luciferase, and Nanoluc luciferase. In some embodiments, the luminescent protein is Gaussia luciferase. In some embodiments, the luminescent protein is superfolder GFP (sfGFP). In some embodiments, the luminescent protein is Renilla luciferase. In some embodiments, the luminescent protein is Nanoluc luciferase. In some embodiments, the reporter molecule is a fluorescent protein including, but not limited to, GFP, YFP, mCherry, dsRed, VENUS, or CFP, and a blue fluorescent protein, or any analog thereof. In some embodiments, the fluorescent protein is GFP. In some embodiments, the fluorescent protein is YFP. In some embodiments, the fluorescent protein is mCherry. In some embodiments, the fluorescent protein is dsRed. In some embodiments, the fluorescent protein is VENUS. In some embodiments, the fluorescent protein is CFP. In some embodiments, the fluorescent protein is a blue fluorescent protein.

In some embodiments, when the luminescent protein is Gaussia luciferase, one of the first fragment of Gaussia luciferase and second fragment of Gaussia luciferase comprises MKPTENNEDFNIVAVASNFATTDLDADRGKLPGKKLPLEVLKEMEANARKAGCTR GCLICLSHIKCTPKMKKFIPGRCHTYEGDKESAQGGIG (SEQ ID NO:5), and the other of the first fragment of Gaussia luciferase and second fragment of Gaussia luciferase comprises EAIVDIPEIPGFKDLEPMEQFIAQVDLCVDCTTGCLKGLANVQCSDLLKKW LPQRCATFASKIQGQVDKIKGAGGD (SEQ ID NO:6).

In some embodiments, when the luminescent protein is sfGFP, one of the first fragment of sfGFP and second fragment of sfGFP comprises MRKGEELFTGVVPILVELD GDVNGHKFSVRGEGEGDATNGKLTLKFICTTGKLPVPWPTLVTTLTYGVQCFARYP DHMKQHDFFKSAMPEGYVQERTISFKDDGTYKTRAEVKFEGDTLVNRIELKGIDFKE DGNILGHKLEYNFNSHNVYITADKQ (SEQ ID NO:7) or MSKGEELFTGVVPILVELDG DVNGHKFSVRGEGEGDATNGKLTLKFICTTGKLPVPWPTLVTTLTYGVQCFSRYPD HMKRHDFFKSAMPEGYVQERTISFKDDGTYKTRAEVKFEGDTLVNRIELKGIDFKED GNILGHKLEYNFNSHNVYITADKQ (SEQ ID NO:22), and the other of the first fragment of sfGFP and second fragment of sfGFP comprises KNGIKANFKIRHNVEDGSVQLADHY QQNTPIGDGPVLLPDNHYLSTQSVLSKDPNEKRDHMVLLEFVTAAGITHGMDELYK (SEQ ID NO:8).

In some embodiments, when the luminescent protein is Renilla luciferase, one of the first fragment of Renilla luciferase and second fragment of Renilla luciferase comprises MAS KVYDPEQRKRMITGPQWWARCKQMNVLDSFINYYDSEKHAENAVIFLHGNAASSY LWRHVVPHIEPVARCIIPDLIGMGKSGKSGNGSYRLLDHYKYLTAWFELLNLPKKIIF VGHDWGACLAFHYSYEHQDKIKAIVHAESVVDVIESWDEWPDIEEDIALIKSEEGEK MVLENNFFVETMLPSKIMRKLEPEEFAAYLEPFKEKGEVRRPTLSWPREIPLVKGG (SEQ ID NO:9), and the other of the first fragment of Renilla luciferase and second fragment of Renilla luciferase comprises KPDVVQIVRNYNAYLRASDDLPKMFIESDPGFFSNAIV EGAKKFPNTEFVKVKGLHFSQEDAPDEMGKYIKSFVERVLKNEQ (SEQ ID NO: 10).

In some embodiments, when the luminescent protein is Nanoluc luciferase, one of the first fragment of Nanoluc luciferase and second fragment of Nanoluc luciferase comprises MFTLEDFVGDWEQTAAYNLDQVLEQGGVSSLLQNLAVSVTPIQRIVRSGENALKIDI HVIIPYEGLSADQMAQIEEVFKVVYPVDDHHFKVILPYGTLVIDGVTPNMLNYFGRP YEGIAVFDGKKITVTGTLWNGNKIIDERLITPDGSMLFRVTINS (SEQ ID NO:34), and the other of the first fragment of Nanoluc luciferase and second fragment of Nanoluc luciferase comprises VSGWRLFKKIS (SEQ ID NO:35) or VTGYRLFEEIL (SEQ ID NO:36).

In some embodiments, the full-length Nanoluc/Ser-Gly linkers/FKBP/6H construct comprises MFTLEDFVGDWEQTAAYNLDQVLEQGGVSSLLQNLAVSVTPIQRIVRSGE NALKIDIHVIIPYEGLSADQMAQIEEVFKVVYPVDDHHFKVILPYGTLVIDGVTPNML NYFGRPYEGIAVFDGKKITVTGTLWNGNKIIDERLITPDGSMLFRVTINGVTGWRLCE RILASGGGGSGGGGSGGGSGVQVETISPGDGRTFPKRGQTVVVHYTGMLEDGKKV DSSRDRNKPFKFMLGKQEVIRGWEEGVAQMSVGQRAKLTISPDYAYGATGHPGIIPP HATLVFDVELLKLEGGSGHHHHHH* (SEQ ID NO:37) (which contains the C22V mutation into FKBP). In some embodiments, the N-terminal Nanoluc split-protein (SP)/Ser-Gly linkers/FKBP/6H construct comprises MFTLEDFVGDWEQTAAYNLDQVLEQGGVS SLLQNLAVSVTPIQRIVRSGENALKIDIHVIIPYEGLSADQMAQIEEVFKVVYPVDDHH FKVILPYGTLVIDGVTPNMLNYFGRPYEGIAVFDGKKITVTGTLWNGNKIIDERLITPD GSMLFRVTINSGGGGSGGGGSGGGSGVQVETISPGDGRTFPKRGQTVVVHYTGML EDGKKVDSSRDRNKPFKFMLGKQEVIRGWEEGVAQMSVGQRAKLTISPDYAYGAT GHPGIIPPHATLVFDVELLKLEGGSGHHHHHH* (SEQ ID NO:38) (which contains the C22V mutation into FKBP). In some embodiments, the 6H/Ser-Gly linkers/FKBP/High-affinity C-terminal Nanoluc fragment construct comprises MHHHHHHGGSGGVQVETISP GDGRTFPKRGQTVVVHYTGMLEDGKKVDSSRDRNKPFKFMLGKQEVIRGWEEGVA QMSVGQRAKLTISPDYAYGATGHPGIIPPHATLVFDVELLKLESGGGGSGGGGSGG GSVSGWRLFKKIS* (SEQ ID NO:39) (which contains the C22V mutation into FKBP). In some embodiments, the 6H/Ser-Gly linkers/FKBP/Low-affinity C-terminal Nanoluc fragment construct comprises MHHHHHHGGSGGVQVETISPGDGRTFPKRGQTVVVH YTGMLEDGKKVDSSRDRNKPFKFMLGKQEVIRGWEEGVAQMSVGQRAKLTISPDY AYGATGHPGIIPPHATLVFDVELLKLESGGGGSGGGGSGGGSVTGYRLFEEIL* (SEQ ID NO:40) (which contains the C22V mutation into FKBP).

In any of the embodiments described herein, the amount of reporter molecule activity is detected.

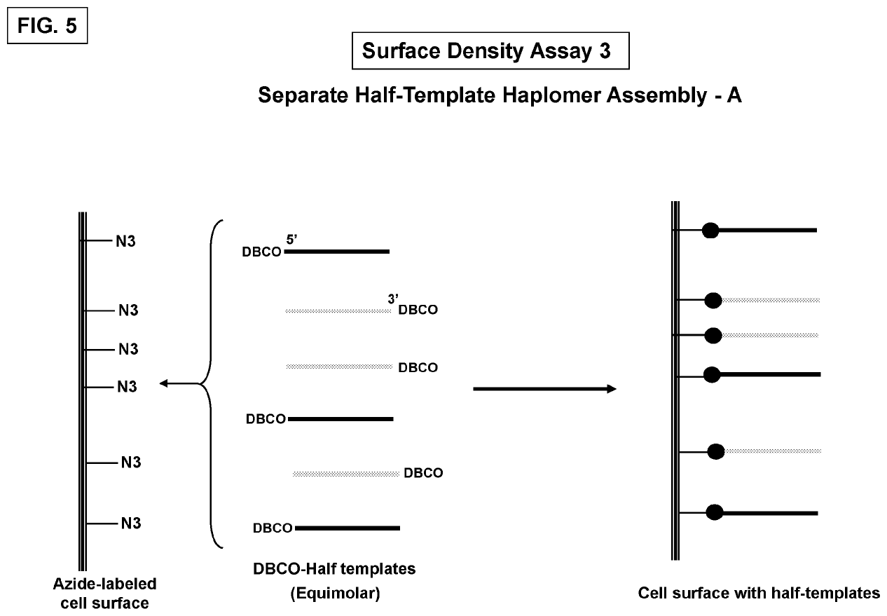

### B) Separate Half-Template Haplomer Assembly Assay (FIGS. 5 and 6)

In related embodiments, a cis vs. trans-complementation is also employed for assessing surface molecular density, by means of using two separate half-templates, each equipped with DBCO moieties appended to the 5′-end of one and the 3′-end of the second (see, FIG. 5). After surface placement of equimolar mixes of such DBCO-modified half-templates, Gaussia haplomer complementation is constrained such that only trans-interactions can result in the formation functional reporter product (see, FIG. 6). In the same manner as for the masked template approach, the efficiency of such trans-interactions are directly related to labeled glycan surface densities. Placement of full-length DBCO-templates on parallel cell preparations can likewise be used to yield a cis-trans complementation activity ratio, as the best gauge of the density factor of interest.

The present disclosure also provides methods of determining surface glycan density of a cell, the method comprising the steps: a) contacting the cell with an azide-modified sugar; b) contacting the cell with a first nucleic acid template comprising a 5′-azide reactive molecule that is chemically reactable with an azide group, wherein the nucleic acid template has a first haplomer hybridization region; c) contacting the cell with a second nucleic acid template comprising a 3′-azide reactive molecule that is chemically reactable with an azide group, wherein the nucleic acid template has a second haplomer hybridization region; d) contacting the cell with a first haplomer comprising: i) a first oligonucleotide conjugated to a first ligand, wherein the first oligonucleotide is complementary to the first haplomer hybridization region; and ii) a first fragment of a reporter molecule conjugated to a first ligand binding domain; wherein the first ligand and the first ligand binding domain associate with one another; e) contacting the cell with a second haplomer comprising: i) a second oligonucleotide conjugated to a second ligand, wherein the second oligonucleotide is complementary to the second haplomer hybridization region; and ii) a second fragment of the reporter molecule conjugated to a second ligand binding domain; wherein the second ligand and the second ligand binding domain associate with one another; whereby, upon hybridization of the first oligonucleotide to the first haplomer hybridization region and the second oligonucleotide to the second haplomer hybridization region, and refolding of the first fragment of the reporter molecule with the second fragment of the reporter molecule, a functional reporter molecule is produced; and f) detecting the amount of reporter molecule activity.

In any of the embodiments described herein, the cell or cells are contacted with an azide-modified sugar. In any of the embodiments described herein, the cell can be any desired target cell. In some embodiments, the cell is a virus infected cell, a tumor cell, a cell infected with a microbe, or a cell that produces a molecule that leads to a disease, such as a cell that produces an antibody that induces allergy, anaphylaxis, or autoimmune disease, or a cytokine that mediates a disease. The cells can be cells of the immune system that are contributing to autoimmunity such as cells of the adaptive or innate immune systems, transplant rejection, or an allergic response. The cells described herein can be contacted with any of the azide-modified sugars described herein either in vitro or in vivo.

In any of the embodiments described herein, the azide-modified sugar is azido-N-acetylmannosamine (AzNAM), azido-N-acetylglucosamine (AzGlcNAc), azido-N-acetylgalactosamine (AGalNAc), or azido-N-acetylneuraminic acid (AzNANA). In some embodiments, the azide-modified sugar is AzNAM. In some embodiments, the azide-modified sugar is AzGlcNAc. In some embodiments, the azide-modified sugar AGalNAc. In some embodiments, the azide-modified sugar is AzNANA. In any of the embodiments described herein, the azide-modified sugar is acetylated at 1, 2, 3, or 4 positions. In some embodiments, the azide-modified sugar is acetylated at 1 position. In some embodiments, the azide-modified sugar is acetylated at 2 positions. In some embodiments, the azide-modified sugar is acetylated at 3 positions. In some embodiments, the azide-modified sugar is acetylated at 4 positions.

In any of the embodiments described herein, the cell or cells are contacted with a first nucleic acid template comprising a 5′-azide reactive molecule that is chemically reactable with an azide group, wherein the nucleic acid template has a first haplomer hybridization region. The cell or cells are also contacted with a second nucleic acid template comprising a 3′-azide reactive molecule that is chemically reactable with an azide group, wherein the nucleic acid template has a second haplomer hybridization region. In any of the embodiments described herein, the azide reactive molecule is dibenzocyclooctyne (DBCO), bicyclo[6.1.0]nonyne (BCN), methyltetrazine, or trans-cyclooctene (TCO). In some embodiments, the azide reactive molecule is DBCO. In some embodiments, the azide reactive molecule is BCN. In some embodiments, the azide reactive molecule is methyltetrazine. In some embodiments, the azide reactive molecule is TCO. In addition, the nucleic acid template has a first haplomer hybridization region and a second haplomer hybridization region.

In any of the embodiments described herein, the cell or cells are contacted with a first haplomer comprising: i) a first oligonucleotide conjugated to a first ligand, wherein the first oligonucleotide is complementary to the first haplomer hybridization region of the first nucleic acid template; and ii) a first fragment of a reporter molecule conjugated to a first ligand binding domain, wherein the first ligand and first ligand binding domain associate with one another.

In any of the embodiments described herein, the cell or cells are contacted with a second haplomer comprising: i) a second oligonucleotide conjugated to a second ligand, wherein the second oligonucleotide is complementary to the second haplomer hybridization region of the second nucleic acid template; and ii) a second fragment of the reporter molecule conjugated to a second ligand binding domain, wherein the second ligand and the second ligand binding domain associate with one another. Upon hybridization of the first oligonucleotide to the first haplomer hybridization region of the first nucleic acid template and the second oligonucleotide to the second haplomer hybridization region of the first nucleic acid template, and refolding of the first fragment of the reporter molecule with the second fragment of the reporter molecule, a functional reporter molecule is produced.

In some embodiments, the first ligand and second ligand are small molecule ligands. In some embodiments, the small molecule ligand is an FKBP-binding compound. In some embodiments, the FKBP-binding compound is chosen from FKM-NHS, FKM-sulfo-NHS, FKM-PEG₁₋₆-NHS, monovalent FKBP Ligand-2 (MFL2), FKM-PEG₁₋₆-MTZ-NHS, and FKM-PEG₁₋₆-TCO-NHS. In some embodiments, the FKBP-binding compound is FKM-NHS. In some embodiments, the FKBP-binding compound is FKM-sulfo-NHS. In some embodiments, the FKBP-binding compound is FKM-PEG₁₋₆-NHS. In some embodiments, the FKBP-binding compound is monovalent FKBP Ligand-2 (MFL2). In some embodiments, the FKBP-binding compound is FKM-PEG₁₋₆-MTZ-NHS. In some embodiments, the FKBP-binding compound is FKM-PEG₁₋₆-TCO-NHS. For example, some embodiments use modifications of the FKM-NHS series of compounds with selectively-reactive moiety side chains. In some embodiments, FKM-PEG₁₋₆-NHS is modified at the C2 position of the PEG chain with a methyltetrazine group (FKM-PEG₁₋₆-MTZ-NHS), or a trans-cyclooctene group (FKM-PEG₁₋₆-TCO-NHS)

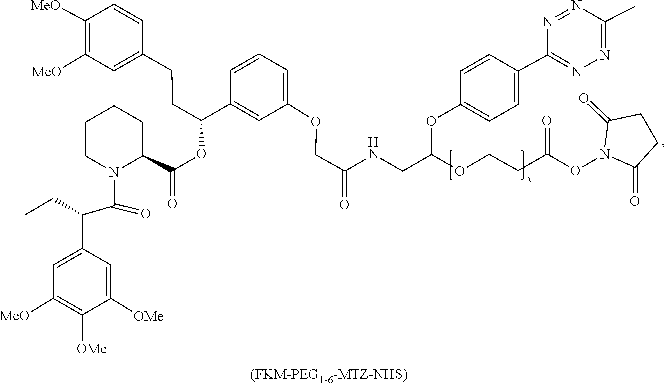

where x is from 1 to 6; and FKM-PEG3-TCO-NHS is

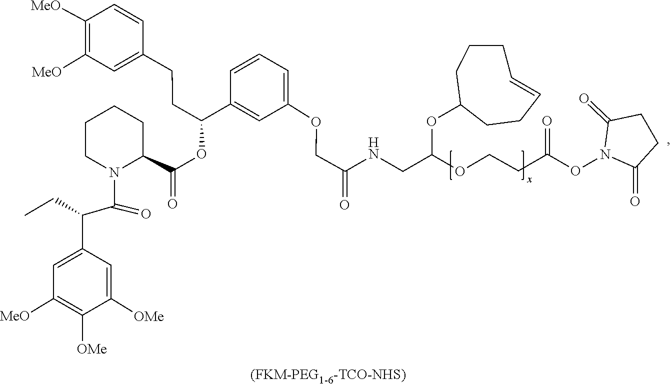

where x is from 1 to 6.

In some embodiments, when the first ligand and second ligand are small molecule ligands as set forth above, the first ligand binding domain and second ligand binding domains are FKBP domains or FRB domains. In some embodiments, the FKBP domain is a mutant FKBP domain. In some embodiments, the mutant FKBP domain is the F36V FKBP mutant domain comprising the amino acid sequence GVQVETISPGDGRTFPKRGQTCVVHYTGM LEDGKKVDSSRDRNKPFKFMLGKQEVIRGWEEGVAQMSVGQRAKLTISPDYAYGA TGHPGIIPPHATLVFDVELLKLE (SEQ ID NO:1) or MGVQVETISPGDGRTFPKRGQTC VVHYTGMLEDGKKVDSSRDRNKPFKFMLGKQEVIRGWEEGVAQMSVGQRAKLTIS PDYAYGATGHPGIIPPHATLVFDVELLKLE (SEQ ID NO:2). In some embodiments, the mutant FKBP domain comprises a C22S, C22A, or C22V substitution. In some embodiments, the FRB domain comprises a C61S, C61A, or C61V substitution.

In some embodiments, the first ligand and first ligand binding domain and/or the second ligand and second ligand binding domain are small interactive protein domain pairs. In some embodiments, the relatively small mutually interactive protein domains (e.g., fragments of proteins) are leucine zippers. Suitable examples of interactive protein domains are the c-jun and c-fos zipper domains, which generally are polypeptides of less than 50 amino acid residues, including helix-initiating and helix-terminating segments. While c-jun can form homodimers, c-fos cannot; and c-fos:c-jun heterodimers are significantly more stable than c-jun:c-jun homodimers. In some embodiments, the small interactive protein domain pairs are chosen from jun/fos, mad/max, myc/max, and NZ/CZ domains. In some embodiments, the small interactive protein domain pair is jun/fos. In some embodiments, the small interactive protein domain pair is mad/max. In some embodiments, the small interactive protein domain pair is myc/max. In some embodiments, the small interactive protein domain pair is NZ/CZ domains. In some embodiments, the interactive protein domain pair is antiparallel zippers, such as that from Thermus thermophilus seryl-tRNA synthetase.

In embodiments using small interactive protein domains as ligands, the polarity of the conjugation of the small domain tag should be taken into account. This can be exemplified with the particular embodiments using fos-jun heterodimerization, where the leucine zipper interaction occurs with a parallel orientation. If haplomers have appended c-Jun tags such that their c-Jun helices are in a parallel orientation following hybridization, then subsequent complex formation with c-fos will orient in a parallel sense; the reverse situation may disfavor dimerization between the protein segments of interest. However, for certain other applications, an antiparallel orientation can be beneficial. For this reason, it is advantageous if strategies exist for conjugating 5′ or 3′ polynucleotide ends with small protein tags by either their N- or C-termini.

In some embodiments, a sequence for making N-terminal conjugates of c-jun can be a 47-mer, where the N-terminal cysteine is shown, and the bold sequences denote helical boundaries: CSGGASLERIARLEEKVKTLKAQNSELASTANMLREQVAQLKQKGAP (SEQ ID NO:11). In some embodiments, a sequence for making C-terminal conjugates of c-jun can be a 49-mer, where the C-terminal cysteine is shown, and the bold sequences denote helical boundaries: SGASLERIARLEEKVKTLKAQNSELASTANMLREQVAQLKQKGA PSGGC (SEQ ID NO:12). In some embodiments, mutants of c-jun are used that cannot form homodimers, but which can still heterodimerize with c-fos. Such modified sequences with N-terminal cysteine residues include, but are not limited to: CSGGASLERIARLEEKVKSFKA QNSENASTANMLREQVAQLKQKGAP (SEQ ID NO:13), where bold residues denote changes from wild-type, and double-underlined sequences denote helical boundaries. Such modified sequences with C-terminal cysteine residues include, but are not limited to: SGAS LERIARLEEKVKSFKAQNSENASTANMLREQVAQLKQKGAPSGGC (SEQ ID NO:14), where bold residues denote changes from wild-type, and double-underlined sequences denote helical boundaries. Additional c-jun sequences include CSGASLERIARLEEKVKSFKAQNS ENASTANMLREQVAQLKQKGAP (SEQ ID NO:15) and GASLERIARLEEKVKTLKAQ NSELASTANMLREQVAQLKQKGAPSGGC (SEQ ID NO:16).

In some embodiments, a sequence for making conjugates of c-fos can be a 41-mer, where the bold sequences denote helical boundaries: ASRELTDTLQAETDQLEDEKSALQ TEIANLLKEKEKLEGAP (SEQ ID NO:17). In some embodiments, the c-fos domain comprises the amino acid sequence ASRETDTLQAETDQLEDEKSALQTEIANLLKEKEK LEGAP (SEQ ID NO:18) or SGASRELTDTLQAETDQLEDEKSALQTEIANLLKEKEKLE GAP (SEQ ID NO:19). Additional extended serine-glycine linkers can be inserted between the c-fos sequence and the sequence to which it is linked.

In some embodiments, the NZ domain comprises the amino acid sequence ALKKEL QANKKELAQLKWELQALKKELAQ (SEQ ID NO:20), and the CZ domain comprises the amino acid sequence EQLEKKLQALEKKLAQLEWKNQALEKKLAQ (SEQ ID NO:21).

In any of the embodiments described herein, the reporter molecule is chosen from a luminescent protein, murine dihydrofolate reductase (DHFR), S. cerevisiae ubiquitin, β-lactamase, and Herpes simplex virus type 1 thymidine kinase. In some embodiments, the reporter molecule is a luminescent protein. In some embodiments, the reporter molecule is DHFR. In some embodiments, the reporter molecule is S. cerevisiae ubiquitin. In some embodiments, the reporter molecule is β-lactamase. In some embodiments, the reporter molecule is a Herpes simplex virus type 1 thymidine kinase. In any of the embodiments described herein, the reporter molecule can also be chosen from a choramphenicol acetyl transferase, a β-galactosidase, and a β-glucuronidase. In some embodiments, the reporter molecule is a choramphenicol acetyl transferase. In some embodiments, the reporter molecule is a β-galactosidase. In some embodiments, the reporter molecule is a 8-glucuronidase.

In some embodiments, when the reporter molecule is DHFR, one of the first fragment of the reporter molecule and the second fragment of the reporter molecule comprises amino acids 1-105 of DHFR, and the other of the first fragment of the reporter molecule and second fragment of the reporter molecule comprises amino acids 106-186 of DHFR.

In some embodiments, when the reporter molecule is S. cerevisiae ubiquitin, one of the first fragment of the reporter molecule and the second fragment of the reporter molecule comprises amino acids 1-34 (MQIFVKTLTGKTITLEVESSDTIDNVKSKIQDKE; SEQ ID NO:3), and the other of the first fragment of the reporter molecule and second fragment of the reporter molecule comprises amino acids 35-76 (GIPPDQQRLIFAGKQLEDGRTLSDY NIQKESTLHLVLRLRGG; SEQ ID NO:4).

In some embodiments, when the reporter molecule is β-lactamase, one of the first fragment of the reporter molecule and the second fragment of the reporter molecule comprises amino acids 25-197 of β-lactamase, and the other of the first fragment of the reporter molecule and second fragment of the reporter molecule comprises amino acids 198-286 of β-lactamase.

In some embodiments, when the reporter molecule is Herpes simplex virus type 1 thymidine kinase, one of the first fragment of the reporter molecule and the second fragment of the reporter molecule comprises amino acids 1-265 of Herpes simplex virus type 1 thymidine kinase, and the other of the first fragment of the reporter molecule and second fragment of the reporter molecule comprises amino acids 266-376 of Herpes simplex virus type 1 thymidine kinase.

In some embodiments, the reporter molecule is a luminescent protein including, but not limited to, Gaussia luciferase, superfolder GFP (sfGFP), Renilla luciferase, and Nanoluc luciferase. In some embodiments, the luminescent protein is Gaussia luciferase. In some embodiments, the luminescent protein is superfolder GFP (sfGFP). In some embodiments, the luminescent protein is Renilla luciferase. In some embodiments, the luminescent protein is Nanoluc luciferase. In some embodiments, the reporter molecule is a fluorescent protein including, but not limited to, GFP, YFP, mCherry, dsRed, VENUS, or CFP, and a blue fluorescent protein, or any analog thereof. In some embodiments, the fluorescent protein is GFP. In some embodiments, the fluorescent protein is YFP. In some embodiments, the fluorescent protein is mCherry. In some embodiments, the fluorescent protein is dsRed. In some embodiments, the fluorescent protein is VENUS. In some embodiments, the fluorescent protein is CFP. In some embodiments, the fluorescent protein is a blue fluorescent protein.

In some embodiments, when the luminescent protein is Gaussia luciferase, one of the first fragment of Gaussia luciferase and second fragment of Gaussia luciferase comprises MKPTENNEDFNIVAVASNFATTDLDADRGKLPGKKLPLEVLKEMEANARKAGCTR GCLICLSHIKCTPKMKKFIPGRCHTYEGDKESAQGGIG (SEQ ID NO:5), and the other of the first fragment of Gaussia luciferase and second fragment of Gaussia luciferase comprises EAIVDIPEIPGFKDLEPMEQFIAQVDLCVDCTTGCLKGLANVQCSDLLKKW LPQRCATFASKIQGQVDKIKGAGGD (SEQ ID NO:6).

In some embodiments, when the luminescent protein is sfGFP, one of the first fragment of sfGFP and second fragment of sfGFP comprises MRKGEELFTGVVPILVELD GDVNGHKFSVRGEGEGDATNGKLTLKFICTTGKLPVPWPTLVTTLTYGVQCFARYP DHMKQHDFFKSAMPEGYVQERTISFKDDGTYKTRAEVKFEGDTLVNRIELKGIDFKE DGNILGHKLEYNFNSHNVYITADKQ (SEQ ID NO:7) or MSKGEELFTGVVPILVELDG DVNGHKFSVRGEGEGDATNGKLTLKFICTTGKLPVPWPTLVTTLTYGVQCFSRYPD HMKRHDFFKSAMPEGYVQERTISFKDDGTYKTRAEVKFEGDTLVNRIELKGIDFKED GNILGHKLEYNFNSHNVYITADKQ (SEQ ID NO:22), and the other of the first fragment of sfGFP and second fragment of sfGFP comprises KNGIKANFKIRHNVEDGSVQLADHY QQNTPIGDGPVLLPDNHYLSTQSVLSKDPNEKRDHMVLLEFVTAAGITHGMDELYK (SEQ ID NO:8).

In some embodiments, when the luminescent protein is Renilla luciferase, one of the first fragment of Renilla luciferase and second fragment of Renilla luciferase comprises MAS KVYDPEQRKRMITGPQWWARCKQMNVLDSFINYYDSEKHAENAVIFLHGNAASSY LWRHVVPHIEPVARCIIPDLIGMGKSGKSGNGSYRLLDHYKYLTAWFELLNLPKKIIF VGHDWGACLAFHYSYEHQDKIKAIVHAESVVDVIESWDEWPDIEEDIALIKSEEGEK MVLENNFFVETMLPSKIMRKLEPEEFAAYLEPFKEKGEVRRPTLSWPREIPLVKGG (SEQ ID NO:9), and the other of the first fragment of Renilla luciferase and second fragment of Renilla luciferase comprises KPDVVQIVRNYNAYLRASDDLPKMFIESDPGFFSNAIV EGAKKFPNTEFVKVKGLHFSQEDAPDEMGKYIKSFVERVLKNEQ (SEQ ID NO: 10).

In some embodiments, when the luminescent protein is Nanoluc luciferase, one of the first fragment of Nanoluc luciferase and second fragment of Nanoluc luciferase comprises MFTLEDFVGDWEQTAAYNLDQVLEQGGVSSLLQNLAVSVTPIQRIVRSGENALKIDI HVIIPYEGLSADQMAQIEEVFKVVYPVDDHHFKVILPYGTLVIDGVTPNMLNYFGRP YEGIAVFDGKKITVTGTLWNGNKIIDERLITPDGSMLFRVTINS (SEQ ID NO:34), and the other of the first fragment of Nanoluc luciferase and second fragment of Nanoluc luciferase comprises VSGWRLFKKIS (SEQ ID NO:35) or VTGYRLFEEIL (SEQ ID NO:36).

In some embodiments, the full-length Nanoluc/Ser-Gly linkers/FKBP/6H construct comprises MFTLEDFVGDWEQTAAYNLDQVLEQGGVSSLLQNLAVSVTPIQRIVRSGE NALKIDIHVIIPYEGLSADQMAQIEEVFKVVYPVDDHHFKVILPYGTLVIDGVTPNML NYFGRPYEGIAVFDGKKITVTGTLWNGNKIIDERLITPDGSMLFRVTINGVTGWRLCE RILASGGGGSGGGGSGGGSGVQVETISPGDGRTFPKRGQTVVVHYTGMLEDGKKV DSSRDRNKPFKFMLGKQEVIRGWEEGVAQMSVGQRAKLTISPDYAYGATGHPGIIPP HATLVFDVELLKLEGGSGHHHHHH* (SEQ ID NO:37) (which contains the C22V mutation into FKBP). In some embodiments, the N-terminal Nanoluc split-protein (SP)/Ser-Gly linkers/FKBP/6H construct comprises MFTLEDFVGDWEQTAAYNLDQVLEQGGVS SLLQNLAVSVTPIQRIVRSGENALKIDIHVIIPYEGLSADQMAQIEEVFKVVYPVDDHH FKVILPYGTLVIDGVTPNMLNYFGRPYEGIAVFDGKKITVTGTLWNGNKIIDERLITPD GSMLFRVTINSGGGGSGGGGSGGGSGVQVETISPGDGRTFPKRGQTVVVHYTGML EDGKKVDSSRDRNKPFKFMLGKQEVIRGWEEGVAQMSVGQRAKLTISPDYAYGAT GHPGIIPPHATLVFDVELLKLEGGSGHHHHHH* (SEQ ID NO:38) (which contains the C22V mutation into FKBP). In some embodiments, the 6H/Ser-Gly linkers/FKBP/High-affinity C-terminal Nanoluc fragment construct comprises MHHHHHHGGSGGVQVETISP GDGRTFPKRGQTVVVHYTGMLEDGKKVDSSRDRNKPFKFMLGKQEVIRGWEEGVA QMSVGQRAKLTISPDYAYGATGHPGIIPPHATLVFDVELLKLESGGGGSGGGGSGG GSVSGWRLFKKIS* (SEQ ID NO:39) (which contains the C22V mutation into FKBP). In some embodiments, the 6H/Ser-Gly linkers/FKBP/Low-affinity C-terminal Nanoluc fragment construct comprises MHHHHHHGGSGGVQVETISPGDGRTFPKRGQTVVVH YTGMLEDGKKVDSSRDRNKPFKFMLGKQEVIRGWEEGVAQMSVGQRAKLTISPDY AYGATGHPGIIPPHATLVFDVELLKLESGGGGSGGGGSGGGSVTGYRLFEEIL* (SEQ ID NO:40) (which contains the C22V mutation into FKBP).

In any of the embodiments described herein, the amount of reporter molecule activity is detected.

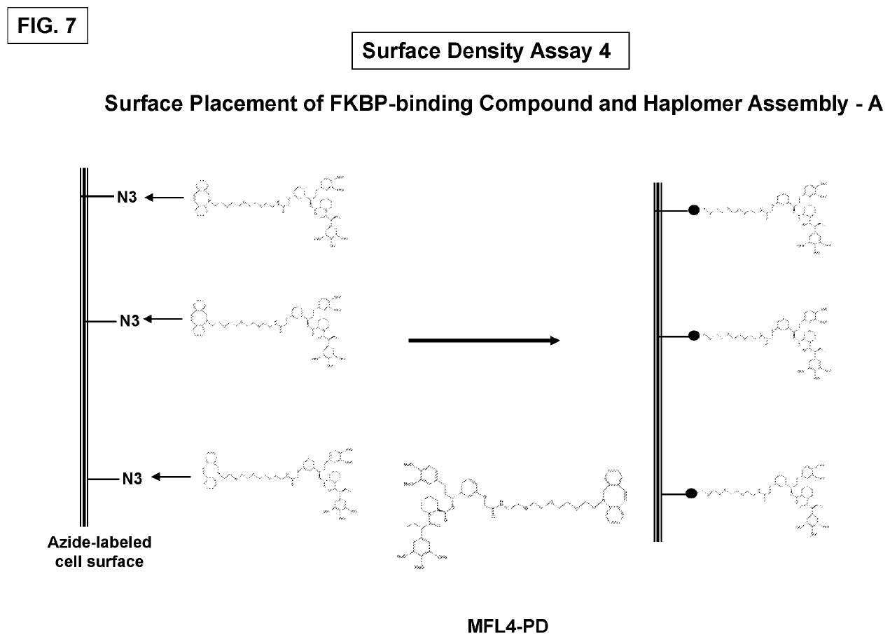

### C) Surface Placement of FKBP-Binding Compounds and Haplomer Assembly Assay (FIGS. 7-9)

In other embodiments for assessing surface molecular density, cell surfaces bearing metabolically-placed azide groups are treated with a bifunctional compound bearing a click-reactive moiety (i.e., an azide-reactive molecule) and a moiety binding to the FKBP domain including, but not limited to, the compounds MFL3-D, MFL4-PD, and MFL5-PBCN. Such bifunctional compounds can be used to react with surface azides and thereby position the FKBP-binding moiety on cell surfaces (shown with the representative MFL4-PD bearing a DBCO group; see, FIG. 7).

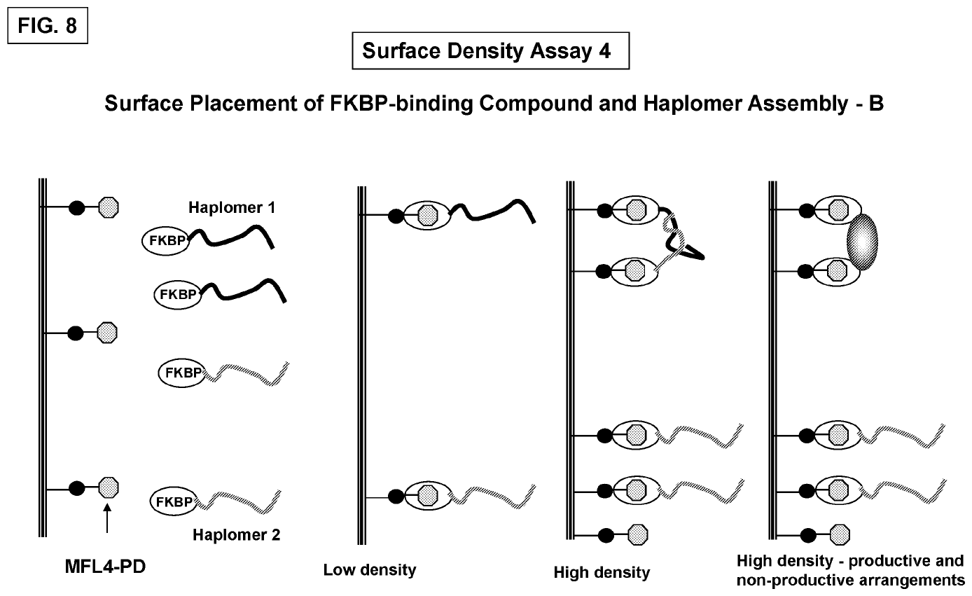

Subsequent to the decoration of a cell surface with the FKBP-binding moiety, cells can be treated with two separate split-protein haplomers where each is fused with the FKBP domain including, but not limited to, variants of FKBP where the cysteine at position 22 is replaced by a valine residue (PCT Publication WO 18/094195). At conditions of low glycan density, surface haplomers are infrequently in close proximity, whereas the frequency of haplomer association rises in proportion to density (see, FIG. 8; as associated with surface azide labeling). Close associations between only two N-terminal or two C-terminal haplomers are non-productive, but the higher the surface labeling, the higher the probability of productive associations occurring (see, FIG. 8), where these are measurable through a reporter assay such as Gaussia luminescence.

The present disclosure provides methods of determining surface glycan density of a cell, the method comprising the steps: a) contacting the cell with an azide-modified sugar; b) contacting the cell with a bifunctional compound, wherein the bifunctional compound comprises an azide reactive molecule that is chemically reactable with an azide group, and a small molecule ligand that binds to an FKBP domain or FRB domain, wherein the bifunctional compound associates with a first glycan molecule, and another bifunctional compound associates with a second glycan molecule; c) contacting the cell with a first haplomer comprising an FKBP domain or FRB domain conjugated to a first fragment of a reporter molecule; d) contacting the cell with a second haplomer comprising an FKBP domain or FRB domain conjugated to a second fragment of the reporter molecule; whereby, upon association between the FKBP domain or FRB domain of the first haplomer and the small molecule ligand of the bifunctional molecule of a first glycan molecule and the association between the FKBP domain or FRB domain of the second haplomer and the small molecule ligand of the bifunctional molecule of a second glycan molecule in sufficient proximity to the first glycan molecule, and refolding of the first fragment of the reporter molecule with the second fragment of the reporter molecule, a functional reporter molecule is produced; and e) detecting the amount of reporter molecule activity.

In any of the embodiments described herein, the cell or cells are contacted with an azide-modified sugar. In any of the embodiments described herein, the cell can be any desired target cell. In some embodiments, the cell is a virus infected cell, a tumor cell, a cell infected with a microbe, or a cell that produces a molecule that leads to a disease, such as a cell that produces an antibody that induces allergy, anaphylaxis, or autoimmune disease, or a cytokine that mediates a disease. The cells can be cells of the immune system that are contributing to autoimmunity such as cells of the adaptive or innate immune systems, transplant rejection, or an allergic response. The cells described herein can be contacted with any of the azide-modified sugars described herein either in vitro or in vivo.

In any of the embodiments described herein, the cell or cells are contacted with an azide-modified sugar. In any of the embodiments described herein, the azide-modified sugar is azido-N-acetylmannosamine (AzNAM), azido-N-acetylglucosamine (AzGlcNAc), azido-N-acetylgalactosamine (AGalNAc), or azido-N-acetylneuraminic acid (AzNANA). In some embodiments, the azide-modified sugar is AzNAM. In some embodiments, the azide-modified sugar is AzGlcNAc. In some embodiments, the azide-modified sugar AGalNAc. In some embodiments, the azide-modified sugar is AzNANA. In any of the embodiments described herein, the azide-modified sugar is acetylated at 1, 2, 3, or 4 positions. In some embodiments, the azide-modified sugar is acetylated at 1 position. In some embodiments, the azide-modified sugar is acetylated at 2 positions. In some embodiments, the azide-modified sugar is acetylated at 3 positions. In some embodiments, the azide-modified sugar is acetylated at 4 positions.

In any of the embodiments described herein, the cell or cells are contacted with a bifunctional compound. The bifunctional compound comprises an azide reactive molecule that is chemically reactable with an azide group. The bifunctional compound also comprises a small molecule ligand that binds to an FKBP domain or FRB domain. The bifunctional compound associates with a first glycan molecule on the surface of a cell or cells, and another bifunctional compound associates with a second glycan molecule on the surface of a cell or cells.

In some embodiments, the azide reactive molecule is chosen from a cyclooctyne, a norbornene, an oxanorbornadiene, a phosphine, a dialkyl phosphine, a trialkyl phosphine, a phosphinothiol, a phosphinophenol, a cyclooctene, a tetrazine, a tetrazole, and a quadricyclane. In some embodiments, the azide reactive molecule is a cyclooctyne. In some embodiments, the azide reactive molecule is a norbornene. In some embodiments, the azide reactive molecule is an oxanorbornadiene. In some embodiments, the azide reactive molecule is a phosphine. In some embodiments, the azide reactive molecule is a dialkyl phosphine. In some embodiments, the azide reactive molecule is a trialkyl phosphine. In some embodiments, the azide reactive molecule is a phosphinothiol. In some embodiments, the azide reactive molecule is a phosphinophenol. In some embodiments, the azide reactive molecule is a cyclooctene. In some embodiments, the azide reactive molecule is a tetrazine. In some embodiments, the azide reactive molecule is a tetrazole. In some embodiments, the azide reactive molecule is a quadricyclane. In some embodiments, the azide reactive molecule is chosen from dibenzocyclooctyne (DBCO), bicyclo[6.1.0]nonyne (BCN), methyltetrazine, and trans-cyclooctene (TCO). In some embodiments, the azide reactive molecule is DBCO. In some embodiments, the azide reactive molecule is BCN. In some embodiments, the azide reactive molecule is methyltetrazine. In some embodiments, the azide reactive molecule is TCO. In addition, the nucleic acid template has a first haplomer hybridization region and a second haplomer hybridization region.

In some embodiments, the azide reactive molecule is chosen from a cyclooctyne, a cyclooctene, and a tetrazine. In some embodiments, the cyclooctyne is dibenzocyclooctyne (DBCO), bicyclo[6.1.0]nonyne (BCN), monofluorinated cyclooctyne, difluorocyclooctyne, dimethoxyazacyclooctyne, dibenzoazacyclooctyne, biarylazacyclooctynone, 2,3,6,7-tetramethoxy-dibenzocyclooctyne, sulfonylated dibenzocyclooctyne, pyrrolocyclooctyne, or carboxymethylmonobenzocyclooctyne. In some embodiments, the cyclooctene is trans-cyclooctene (TCO). In some embodiments, the tetrazine is methyltetrazine, diphenyltetrazine, 3,6-di-(2-pyridyl)-s-tetrazine, 3,6-diphenyl-s-tetrazine, 3-(5-aminopyridin-2-yl)-6-(pyridin-2-yl)-s-tetrazine, or N-benzoyl-3-(5-aminopyridin-2-yl)-6-(pyridin-2-yl)-s-tetrazine.

In some embodiments, the small molecule ligand binds to an FKBP domain. In some embodiments, the small molecule ligand binds to an FRB domain. In some embodiments, the small molecule ligand is chosen from FKM-NHS, FKM-sulfo-NHS, FKM-PEG₁₋₆-NHS, monovalent FKBP Ligand-2 (MFL2), FKM-PEG₁₋₆-MTZ-NHS, and FKM-PEG₁₋₆-TCO-NHS. In some embodiments, the FKBP-binding compound is FKM-NHS. In some embodiments, the FKBP-binding compound is FKM-sulfo-NHS. In some embodiments, the FKBP-binding compound is FKM-PEG₁₋₆-NHS. In some embodiments, the FKBP-binding compound is monovalent FKBP Ligand-2 (MFL2). In some embodiments, the FKBP-binding compound is FKM-PEG₁₋₆-MTZ-NHS. In some embodiments, the FKBP-binding compound is FKM-PEG₁₋₆-TCO-NHS. In some embodiments, the FKBP-binding compound is a compound having the formula

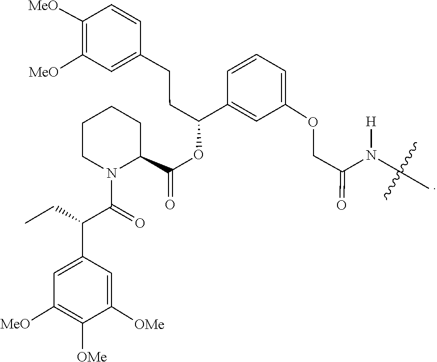 [SMILES: *NC(=O)COc1cccc([C@@H](CCc2ccc(OC)c(OC)c2)OC(=O)[C@@H]2CCCCN2C(=O)[C@@H](CC)c2cc(OC)c(OC)c(OC)c2)c1]

For example, some embodiments use modifications of the FKM-NHS series of compounds with selectively-reactive moiety side chains. In some embodiments, FKM-PEG₁₋₆-NHS is modified at the C2 position of the PEG chain with a methyltetrazine group (FKM-PEG₁₋₆-MTZ-NHS), or a trans-cyclooctene group (FKM-PEG₁₋₆-TCO-NHS)

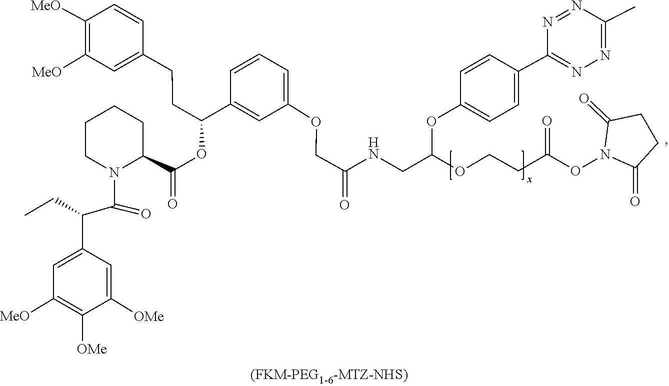

where x is from 1 to 6; and FKM-PEG3-TCO-NHS is

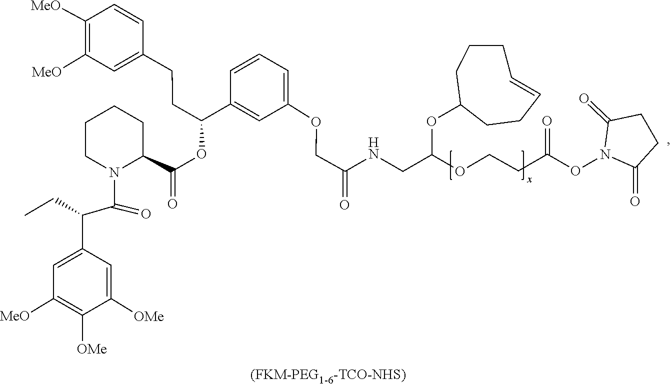

where x is from 1 to 6.

In some embodiments, the bifunctional compound comprises the formula:

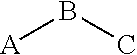

wherein: A is a small molecule ligand that binds to an FKBP domain or FRB domain; B is a chemical linker chosen from an alkyl, an alkenyl, an amide, an ester, a thioester, a ketone, an ether, a thioether, a disulfide, an ethylene glycol unit, a cycloalkyl, a benzyl, a heterocyclic, a maleimidyl, a hydrazone, a urethane, an azole, an imine, a haloalkyl, or a carbamate, or any combination thereof; and C is an azide reactive molecule such as any of those described herein.

In any of the embodiments described herein, B is a chemical linker chosen from an alkyl, an alkenyl, an amide, an ester, a thioester, a ketone, an ether, a thioether, a disulfide, an ethylene glycol unit, a cycloalkyl, a benzyl, a heterocyclic, a maleimidyl, a hydrazone, a urethane, an azole, an imine, a haloalkyl, or a carbamate, or any combination thereof. In some embodiments, the chemical linker is an alkyl. In some embodiments, the chemical linker is an alkenyl. In some embodiments, the chemical linker is an amide. In some embodiments, the chemical linker is an ester. In some embodiments, the chemical linker is a thioester. In some embodiments, the chemical linker is a disulfide. In some embodiments, the chemical linker is a ketone. In some embodiments, the chemical linker is an ether. In some embodiments, the chemical linker is a thioether. In some embodiments, the chemical linker is an ethylene glycol unit. In some embodiments, the chemical linker is a cycloalkyl. In some embodiments, the chemical linker is a benzyl. In some embodiments, the chemical linker is a heterocyclic. In some embodiments, the chemical linker is a maleimidyl. In some embodiments, the chemical linker is a hydrazone. In some embodiments, the chemical linker is a urethane. In some embodiments, the chemical linker is an azole. In some embodiments, the chemical linker is an imine. In some embodiments, the chemical linker is a haloalkyl. In some embodiments, the chemical linker is a carbamate.

In some embodiments, the chemical linker is an alkyl or an ethylene glycol unit. In some embodiments, the chemical linker is an alkyl. In some embodiments, the chemical linker is a C₂-C₁₆alkyl. In some embodiments, the chemical linker is a C₄-C₁₂alkyl or a C₄-C₁₆alkyl. In some embodiments, the chemical linker is a C₄-C₁₀alkyl. In some embodiments, the chemical linker is C₄alkyl or C₁₀alkyl. In some embodiments, the chemical linker is an ethylene glycol unit. In some embodiments, the chemical linker is a polyethylene glycol (PEG). In some embodiments, the PEG is PEG2 to PEG16. In some embodiments, the PEG is PEG2, PEG3, or PEG4.

In some embodiments, the FKBP domain is the FK506-FKBP domain or the mutant (F36V) FKBP domain, the chemical linker is an alkyl or an ethylene glycol unit, and the azide reactive molecule is chosen from a cyclooctyne, a cyclooctene, and a tetrazine.

In some embodiments, the FKBP domain is the FK506-FK1BP domain or the mutant (F36V) FKBP domain, the chemical linker is a C₂-C₁₆alkyl, or a polyethylene glycol which is PEG2 to PEG16, and the azide reactive molecule is DBCO, BCN, TCO, or methyltetrazine.

In some embodiments, the FKBP domain is the mutant (F36V) FKBP domain, the chemical linker is a C₄-C₁₀alkyl or a polyethylene glycol which is PEG2, PEG3, or PEG4, and the azide reactive molecule is DBCO, BCN, TCO, or methyltetrazine.

In some embodiments, the small molecule ligand is

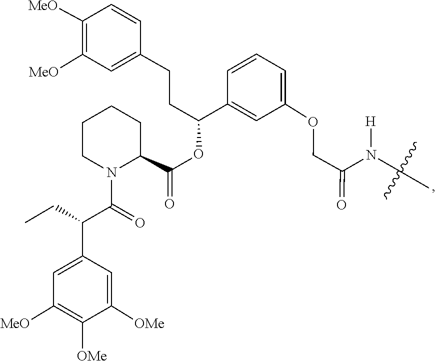 [SMILES: *NC(=O)COc1cccc([C@@H](CCc2ccc(OC)c(OC)c2)OC(=O)[C@@H]2CCCCN2C(=O)[C@@H](CC)c2cc(OC)c(OC)c(OC)c2)c1]

the chemical linker is C₄alkyl, C₁₀alkyl, or PEG3, and the azide reactive molecule is DBCO or BCN.

In some embodiments, the bifunctional compound comprises the formula:

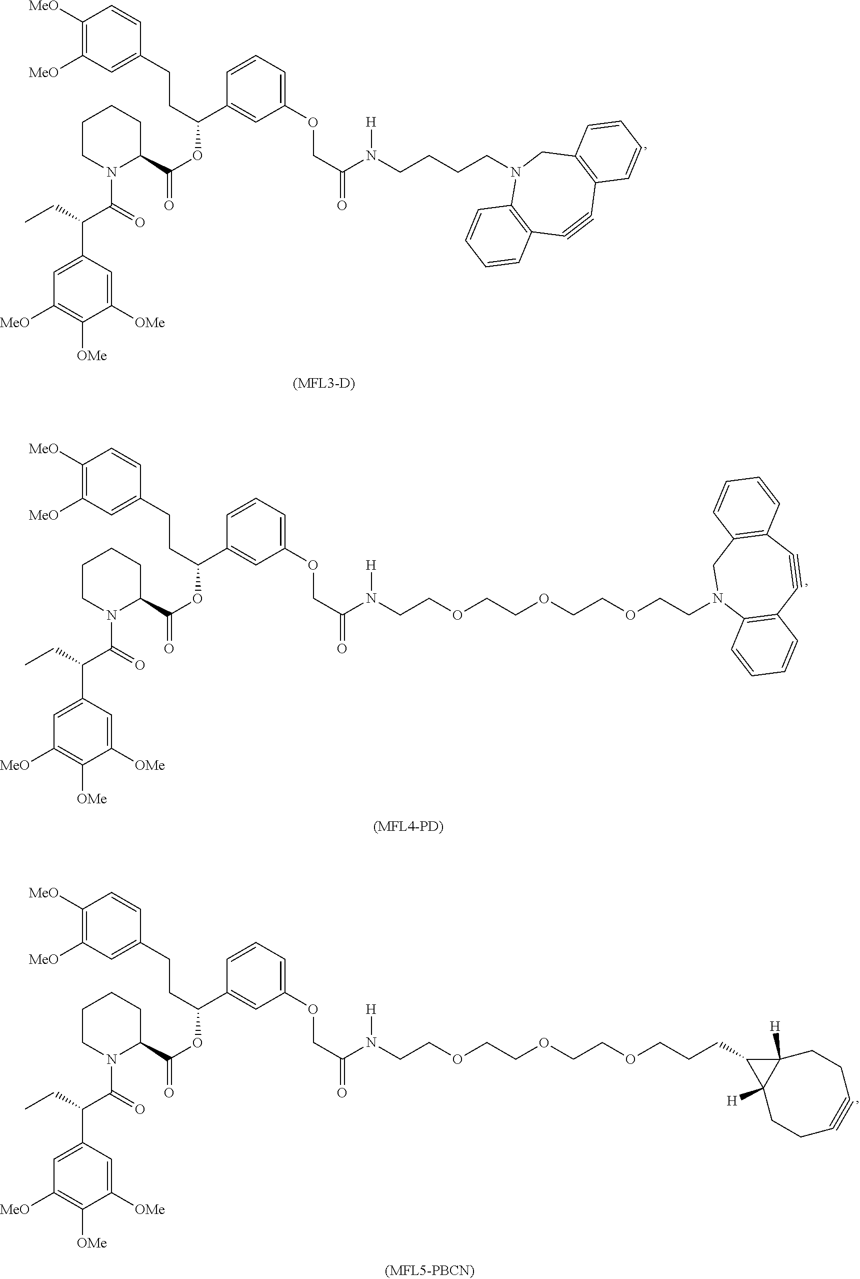 [SMILES: CC[C@H](C(=O)N1CCCC[C@H]1C(=O)O[C@H](CCc1ccc(OC)c(OC)c1)c1cccc(OCC(=O)NCCCCN2Cc3ccccc3C#Cc3ccccc32)c1)c1cc(OC)c(OC)c(OC)c1 ; CC[C@H](C(=O)N1CCCC[C@H]1C(=O)O[C@H](CCc1ccc(OC)c(OC)c1)c1cccc(OCC(=O)NCCOCCOCCOCCN2Cc3ccccc3C#Cc3ccccc32)c1)c1cc(OC)c(OC)c(OC)c1 ; CC[C@H](C(=O)N1CCCC[C@H]1C(=O)O[C@H](CCc1ccc(OC)c(OC)c1)c1cccc(OCC(=O)NCCOCCOCCOCCC[C@@H]2[C@@H]3CCC#CCC[C@@H]32)c1)c1cc(OC)c(OC)c(OC)c1]

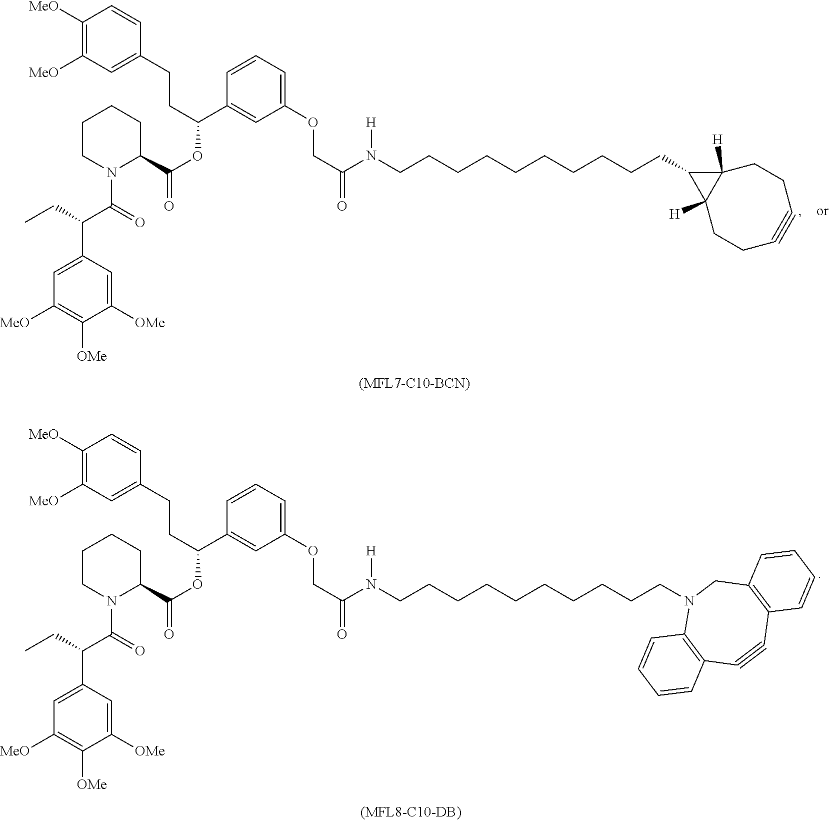 [SMILES: CC[C@H](C(=O)N1CCCC[C@H]1C(=O)O[C@H](CCc1ccc(OC)c(OC)c1)c1cccc(OCC(=O)NCCCCCCCCCC[C@@H]2[C@@H]3CCC#CCC[C@@H]32)c1)c1cc(OC)c(OC)c(OC)c1 ; CC[C@H](C(=O)N1CCCC[C@H]1C(=O)O[C@H](CCc1ccc(OC)c(OC)c1)c1cccc(OCC(=O)NCCCCCCCCCCN2Cc3ccccc3C#Cc3ccccc32)c1)c1cc(OC)c(OC)c(OC)c1]

In any of the embodiments described herein, the cell or cells are contacted with a first haplomer comprising an FKBP domain or FRB domain conjugated to a first fragment of a reporter molecule. The cell or cells are also contacted with a second haplomer comprising an FKBP domain or FRB domain conjugated to a second fragment of the reporter molecule. Upon association between the FKBP domain or FRB domain of the first haplomer and the small molecule ligand of the bifunctional molecule of a first glycan molecule and the association between the FKBP domain or FRB domain of the second haplomer and the small molecule ligand of the bifunctional molecule of a second glycan molecule in sufficient proximity to the first glycan molecule, and refolding of the first fragment of the reporter molecule with the second fragment of the reporter molecule, a functional reporter molecule is produced.

In some embodiments, the FKBP domain of the first and/or second haplomer is the FK506-FKBP domain or the mutant (F36V) FKBP domain. In some embodiments, the FKBP domain of the first and/or second haplomer is the FK506-FKBP domain. In some embodiments, the FKBP domain of the first and/or second haplomer is the mutant (F36V) FK1BP domain. In some embodiments, the F36V FKBP mutant domain comprises the amino acid sequence GVQVETISPGDGRTFPKRGQTCVVHYTGMLEDGKKVDSSRDRNKPFK FMLGKQEVIRGWEEGVAQMSVGQRAKLTISPDYAYGATGHPGIIPPHATLVFDVELL KLE (SEQ ID NO:1) or MGVQVETISPGDGRTFPKRGQTCVVHYTGMLEDGKKVDSSR DRNKPFKFMLGKQEVIRGWEEGVAQMSVGQRAKLTISPDYAYGATGHPGIIPPHATL VFDVELLKLE (SEQ ID NO:2). In some embodiments, the FKBP domain is a mutant FKBP domain that comprises a C22S, C22A, or C22V substitution. In some embodiments, the FRB domain comprises a C61S, C61A, or C61V substitution.

In any of the embodiments described herein, the reporter molecule is a luminescent protein including, but not limited to, Gaussia luciferase, superfolder GFP (sfGFP), Renilla luciferase, and Nanoluc luciferase. In some embodiments, the luminescent protein is Gaussia luciferase. In some embodiments, the luminescent protein is superfolder GFP (sfGFP). In some embodiments, the luminescent protein is Renilla luciferase. In some embodiments, the luminescent protein is Nanoluc luciferase. In some embodiments, the reporter molecule is a fluorescent protein including, but not limited to, GFP, YFP, mCherry, dsRed, VENUS, or CFP, and a blue fluorescent protein, or any analog thereof. In some embodiments, the fluorescent protein is GFP. In some embodiments, the fluorescent protein is YFP. In some embodiments, the fluorescent protein is mCherry. In some embodiments, the fluorescent protein is dsRed. In some embodiments, the fluorescent protein is VENUS. In some embodiments, the fluorescent protein is CFP. In some embodiments, the fluorescent protein is a blue fluorescent protein.

In some embodiments, when the luminescent protein is Gaussia luciferase, one of the first fragment of Gaussia luciferase and second fragment of Gaussia luciferase comprises MKPTENNEDFNIVAVASNFATTDLDADRGKLPGKKLPLEVLKEMEANARKAGCTR GCLICLSHIKCTPKMKKFIPGRCHTYEGDKESAQGGIG (SEQ ID NO:5), and the other of the first fragment of Gaussia luciferase and second fragment of Gaussia luciferase comprises EAIVDIPEIPGFKDLEPMEQFIAQVDLCVDCTTGCLKGLANVQCSDLLKKW LPQRCATFASKIQGQVDKIKGAGGD (SEQ ID NO:6).

In some embodiments, when the luminescent protein is sfGFP, one of the first fragment of sfGFP and second fragment of sfGFP comprises MRKGEELFTGVVPILVELD GDVNGHKFSVRGEGEGDATNGKLTLKFICTTGKLPVPWPTLVTTLTYGVQCFARYP DHMKQHDFFKSAMPEGYVQERTISFKDDGTYKTRAEVKFEGDTLVNRIELKGIDFKE DGNILGHKLEYNFNSHNVYITADKQ (SEQ ID NO:7) or MSKGEELFTGVVPILVELDG DVNGHKFSVRGEGEGDATNGKLTLKFICTTGKLPVPWPTLVTTLTYGVQCFSRYPD HMKRHDFFKSAMPEGYVQERTISFKDDGTYKTRAEVKFEGDTLVNRIELKGIDFKED GNILGHKLEYNFNSHNVYITADKQ (SEQ ID NO:22), and the other of the first fragment of sfGFP and second fragment of sfGFP comprises KNGIKANFKIRHNVEDGSVQLADHY QQNTPIGDGPVLLPDNHYLSTQSVLSKDPNEKRDHMVLLEFVTAAGITHGMDELYK (SEQ ID NO:8).

In some embodiments, when the luminescent protein is Renilla luciferase, one of the first fragment of Renilla luciferase and second fragment of Renilla luciferase comprises MAS KVYDPEQRKRMITGPQWWARCKQMNVLDSFINYYDSEKHAENAVIFLHGNAASSY LWRHVVPHIEPVARCIIPDLIGMGKSGKSGNGSYRLLDHYKYLTAWFELLNLPKKIIF VGHDWGACLAFHYSYEHQDKIKAIVHAESVVDVIESWDEWPDIEEDIALIKSEEGEK MVLENNFFVETMLPSKIMRKLEPEEFAAYLEPFKEKGEVRRPTLSWPREIPLVKGG (SEQ ID NO:9), and the other of the first fragment of Renilla luciferase and second fragment of Renilla luciferase comprises KPDVVQIVRNYNAYLRASDDLPKMFIESDPGFFSNAIV EGAKKFPNTEFVKVKGLHFSQEDAPDEMGKYIKSFVERVLKNEQ (SEQ ID NO:10).

In some embodiments, when the luminescent protein is Nanoluc luciferase, one of the first fragment of Nanoluc luciferase and second fragment of Nanoluc luciferase comprises MFTLEDFVGDWEQTAAYNLDQVLEQGGVSSLLQNLAVSVTPIQRIVRSGENALKIDI HVIIPYEGLSADQMAQIEEVFKVVYPVDDHHFKVILPYGTLVIDGVTPNMLNYFGRP YEGIAVFDGKKITVTGTLWNGNKIIDERLITPDGSMLFRVTINS (SEQ ID NO:34), and the other of the first fragment of Nanoluc luciferase and second fragment of Nanoluc luciferase comprises VSGWRLFKKIS (SEQ ID NO:35) or VTGYRLFEEIL (SEQ ID NO:36).

In some embodiments, the full-length Nanoluc/Ser-Gly linkers/FKBP/6H construct comprises MFTLEDFVGDWEQTAAYNLDQVLEQGGVSSLLQNLAVSVTPIQRIVRSGE NALKIDIHVIIPYEGLSADQMAQIEEVFKVVYPVDDHHFKVILPYGTLVIDGVTPNML NYFGRPYEGIAVFDGKKITVTGTLWNGNKIIDERLITPDGSMLFRVTINGVTGWRLCE RILASGGGGSGGGGSGGGSGVQVETISPGDGRTFPKRGQTVVVHYTGMLEDGKKV DSSRDRNKPFKFMLGKQEVIRGWEEGVAQMSVGQRAKLTISPDYAYGATGHPGIIPP HATLVFDVELLKLEGGSGHHHHHH* (SEQ ID NO:37) (which contains the C22V mutation into FKBP). In some embodiments, the N-terminal Nanoluc split-protein (SP)/Ser-Gly linkers/FKBP/6H construct comprises MFTLEDFVGDWEQTAAYNLDQVLEQGGVS SLLQNLAVSVTPIQRIVRSGENALKIDIHVIIPYEGLSADQMAQIEEVFKVVYPVDDHH FKVILPYGTLVIDGVTPNMLNYFGRPYEGIAVFDGKKITVTGTLWNGNKIIDERLITPD GSMLFRVTINSGGGGSGGGGSGGGSGVQVETISPGDGRTFPKRGQTVVVHYTGML EDGKKVDSSRDRNKPFKFMLGKQEVIRGWEEGVAQMSVGQRAKLTISPDYAYGAT GHPGIIPPHATLVFDVELLKLEGGSGHHHHHH* (SEQ ID NO:38) (which contains the C22V mutation into FKBP). In some embodiments, the 6H/Ser-Gly linkers/FKBP/High-affinity C-terminal Nanoluc fragment construct comprises MHHHHHHGGSGGVQVETISP GDGRTFPKRGQTVVVHYTGMLEDGKKVDSSRDRNKPFKFMLGKQEVIRGWEEGVA QMSVGQRAKLTISPDYAYGATGHPGIIPPHATLVFDVELLKLESGGGGSGGGGSGG GSVSGWRLFKKIS* (SEQ ID NO:39) (which contains the C22V mutation into FKBP). In some embodiments, the 6H/Ser-Gly linkers/FKBP/Low-affinity C-terminal Nanoluc fragment construct comprises MHHHHHHGGSGGVQVETISPGDGRTFPKRGQTVVVH YTGMLEDGKKVDSSRDRNKPFKFMLGKQEVIRGWEEGVAQMSVGQRAKLTISPDY AYGATGHPGIIPPHATLVFDVELLKLESGGGGSGGGGSGGGSVTGYRLFEEIL* (SEQ ID NO:40) (which contains the C22V mutation into FKBP).

In some embodiments, the first haplomer and second haplomer are pre-incubated with excess bifunctional compound prior to their contacting the cells.

In any of the embodiments described herein, the amount of reporter molecule activity is detected.

In any of the embodiments described herein, the nucleic acid molecules described herein can be modified to be nuclease resistant. In some embodiments, the nucleic acid molecule comprises a phosphorothioate backbone, a phosphoramidate backbone, a morpholino backbone, a bridged nucleic acid backbone, a locked nucleic acid (LNA) backbone, or a 2′ modification. In some embodiments, the nucleic acid molecule comprises a phosphorothioate backbone. In some embodiments, the nucleic acid molecule comprises a phosphoramidate backbone. In some embodiments, the nucleic acid molecule comprises a morpholino backbone. In some embodiments, the nucleic acid molecule comprises a bridged nucleic acid backbone. In some embodiments, the nucleic acid molecule comprises an LNA backbone.

In some embodiments, the nucleic acid molecule comprises a 2′ modification. In some embodiments, the 2′ modification is chosen from —O((CH₂)ₙO)ₘCH₃, —O(CH₂)ₙOCH₃, —O(CH₂)ₙNH₂, —O(CH₂)ₙCH₃, —O(CH₂)ₙ—ONH₂, and —O(CH₂)ₙON((CH₂)ₙCH₃))₂, where n and m are, independently, from 0 to about 10. In some embodiments, the 2′ modification is —O((CH₂)ₙO)ₘCH₃, where n and m are, independently, from 0 to about 10. In some embodiments, the 2′ modification is —O(CH₂)ₙOCH₃, where n is from 0 to about 10. In some embodiments, the 2′ modification is —O(CH₂)ₙNH₂, where n is from 0 to about 10. In some embodiments, the 2′ modification is —O(CH₂)ₙCH₃, where n is from 0 to about 10. In some embodiments, the 2′ modification is —O(CH₂)ₙ—ONH₂, where n is from 0 to about 10. In some embodiments, the 2′ modification is —O(CH₂)ₙON((CH₂)ₙCH₃))₂, where each n is, independently, from 0 to about 10. In some embodiments, the 2′ modification is a 2′-O-methyl group.

The nucleic acid molecule sequence can be DNA nucleotides, RNA nucleotides, phosphorothioate-modified nucleotides, phosphoramidate-modified nucleotides, 2-O-alkylated RNA nucleotides, halogenated nucleotides, locked nucleic acid nucleotides (LNA), peptide nucleic acids (PNA), morpholino nucleic acid analogues (morpholinos), pseudouridine nucleotides, xanthine nucleotides, hypoxanthine nucleotides, 2-deoxyinosine nucleotides, bridged nucleic acid backbone, other nucleic acid analogues capable of base-pair formation, or combinations thereof.

The sequence of bases in a nucleic acid molecule can be complementary to a hybridization site on a corresponding nucleic acid molecule, allowing sequence-specific binding of the two nucleic acid molecules. In some embodiments, the hybridization site is selected such that its sequence is not similar to sequences known to be present in non-target nucleic acid templates. In some embodiments, the hybridization site includes one or more mutations found within the nucleic acid template, allowing specific binding of nucleic acid recognition moiety to the nucleic acid template but not to non-target nucleic acids that do not contain the mutation. The binding site on the nucleic acid template can be anywhere from about 5 to about 100 bases in length. In some embodiments, the binding site on the nucleic acid template can be from about 5 to about 50 bases in length. In some embodiments, the binding site on the nucleic acid template can be from about 5 to about 40 bases in length. In some embodiments, the binding site on the nucleic acid template can be from about 10 to about 30 bases in length.

Likewise, the nucleic acid recognition moiety can be a polynucleotide that can bind to the nucleic acid template. The polynucleotide can be from about 5 to about 100 bases in length. In some embodiments, the polynucleotide can be from about 5 to about 50 bases in length. In some embodiments, the polynucleotide can be from about 5 to about 40 bases in length. In some embodiments, the polynucleotide can be from about 10 to about 30 bases in length.

The nucleic acid recognition moiety can also be optimized to provide a desired interaction with the nucleic acid template. The length of the nucleic acid template that the nucleic acid recognition moiety binds can be selected based on chemical properties of the complementary sequence of the nucleic acid template. Such properties include the melting and annealing temperatures of the complementary sequence. The melting temperature, Tₘ, is defined as the temperature in degrees Celsius, at which 50% of all molecules of a given nucleic acid sequence are hybridized into a double strand, and 50% are present as single strands. The annealing temperature is generally 5° C. lower than the melting temperature.

The Tₘ of the complementary sequence of the nucleic acid template can be from about 10° C. below to about 40° C. above the temperature of the conditions in which the haplomer will be used. For example, if haplomers are to be used at 37° C., the nucleic acid recognition moiety can be designed with an expected Tₘ between 27° C. to 77° C. In some embodiments, the haplomers can be used at approximately 37° C., and the Tₘ of the complementary sequence used in the nucleic acid recognition moiety can be designed to be from about 37° C. to about 52° C.

In some embodiments, the nucleic acid recognition moiety can be designed such that the Tₘ to bind the nucleic acid template is substantially different from the Tₘ to bind a similar non-target nucleic acid. For example, the nucleic acid recognition moiety can be designed such that the hybridization site it binds to on a nucleic acid template includes the site of a mutation. In some embodiments, the Tₘ of the nucleic acid recognition moiety binding to the nucleic acid template is at or above the temperature at which the haplomer will be used, while the Tₘ of the nucleic acid recognition moiety binding to the non-target nucleic acid is below the temperature at which the haplomer will be used. The nucleic acid recognition moiety will then bind to the mutant nucleic acid template, but not to the non-target, non-mutant sequence.

Binding or hybridization sites of the nucleic acid recognition moieties of members of a set of corresponding haplomers can be on the same nucleic acid template. In some embodiments, the binding or hybridization sites can be found on the same nucleic acid template but separated by about 0 to about 100 bases on the nucleic acid template. In some embodiments, the binding or hybridization sites can be separated by about 0 to about 30 bases on the nucleic acid template. In some embodiments, the binding or hybridization sites can be separated by distances greater than 30 bases on the same nucleic acid template, but be brought into closer proximity through secondary or tertiary structure formation of the nucleic acid template. In some embodiments, the binding or hybridization sites can be separated by a distance greater than 100 bases and brought into closer proximity through secondary or tertiary structure formation of the nucleic acid template. In some embodiments, the polynucleotide of the haplomer comprises from about 6 to about 20 nucleotide bases. In some embodiments, the polynucleotide of the haplomer comprises from about 8 to about 15 nucleotide bases.

The reporter molecule fragment does not possess the targeted activity or the same level of activity associated with the active reporter molecule. In some instances, the reporter molecule fragment is substantially inactive compared to the active reporter molecule. In some embodiments, the individual reporter molecule fragment can possess separate activity, but binding the reporter molecule fragments together creates an activity not possessed by them individually.

Chemical linkers can also be incorporated into the haplomers. The chemical linkers can be included between any of the moieties. Chemical linkers can optionally connect two or more of the moieties to introduce additional functionality or facilitate synthesis. The chemical linker can be a bond between any of the moieties. The chemical linkers can aid in facilitating spatial separation of the moieties, increasing flexibility of the moieties relative to each other, introducing a cleavage site or modification site to the haplomer, facilitating synthesis of the haplomer, improving physical or functional characteristics (such as solubility, hydrophobicity, charge, cell-permeability, toxicity, biodistribution, or stability) of the haplomer, or any combination of the above. In some embodiments, the chemical linker is derived from a cross-linker that facilitates connecting the haplomer components via bioconjugation chemistry. Due to mild reaction conditions, bioconjugate chemistry approaches can be suitable for ligating biomolecules, such as nucleic acids, peptides, or polysaccharides. Examples include, but are not limited to, chains of one or more of the following: alkyl groups, alkenyl groups, amides, esters, thioesters, ketones, ethers, thioethers, disulfides, ethylene glycol, cycloalkyl groups, benzyl groups, heterocyclic groups, maleimidyl groups, hydrazones, urethanes, azoles, imines, haloalkyl groups, and carbamates, or any combination thereof.

For any of the any of the haplomer polynucleotides described herein, the complementarity with another nucleic acid molecule can be 100%. In some embodiments, one particular nucleic acid molecule can be substantially complementary to another nucleic acid molecule. As used herein, the phrase “substantially complementary” means from 1 to 10 mismatched base positions, from 1 to 9 mismatched base positions, from 1 to 8 mismatched base positions, from 1 to 7 mismatched base positions, from 1 to 6 mismatched base positions, from 1 to 5 mismatched base positions, from 1 to 4 mismatched base positions, from 1 to 3 mismatched base positions, and 1 or 2 mismatched base positions.

In some embodiments, the linkage between two amino acid sequences can comprise a linker. In some embodiments, the linker is a Ser/Gly linker, a Poly-Asparagine linker, or a linker comprising the amino acid sequence AGSSAAGSGS (SEQ ID NO:23). In some embodiments, the Poly-Asparagine linker comprises from about 8 to about 16 asparagine residues. In some embodiments, the Ser/Gly linker comprises GGSGGGSGGGSGGGSGGG (SEQ ID NO:24), GGSGGGSGGGSGGGSGGGSGGG (SEQ ID NO:25), GGSGGGSGGG SGGGSGGGSGGGSGGG (SEQ ID NO:26), SGGGGSGGGGSGGGG (SEQ ID NO:27), SGGGGSGGGGSGGGGSGGGG (SEQ ID NO:28), SGGGGSGGGGSGGGGSGGGGSGG GG (SEQ ID NO:29), SGGGS (SEQ ID NO:30), SGSG (SEQ ID NO:31), SGGGGS (SEQ ID NO:32), or SGSGG (SEQ ID NO:33).

In order that the subject matter disclosed herein may be more efficiently understood, examples are provided below. It should be understood that these examples are for illustrative purposes only and are not to be construed as limiting the claimed subject matter in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1: Labeling Cells with Azide-Modified Sugars

Demonstration of the presence of azides on a cell surface was achieved by treating a cell preparation with a fluorescent azide reactive molecule that reacts only with the azide group, without chemical reactivity with normal biological molecules. Initially, cells were cultured in a suitable culture vessel such that their level of confluency at the time of addition of the azide-modified sugar, AzNAM, was not more than 80%.

In particular, HeLa cells were plated in 6-well plates (2.5×10⁴ cells/well) and incubated for 48 hours in DMEM-10% FBS medium in a standard 5% CO₂ atmosphere. Medium from each well was removed, and each well was washed with 2 ml of phosphate buffered saline (PBS), and fresh DMEM-FBS (1 ml) added. Subsequently, varying amounts of AzNAM (solubilized in DMSO) were added to the wells in small (10 μl) volumes to produce the desired final concentrations in the range 10-125 μM. After an additional 20 hours incubation, the medium in each well was removed, and the wells were washed with 2 ml of PBS, followed by addition of 1 ml of PBS. Then, a fluorescent azide reactive molecule, DBCO-FAM (Broadpharm), was directly added to each well to produce a final concentration of 10 μM.

Plates with added DBCO-FAM were protected from bright light and incubated for 1 hour at room temperature. Supernatants in each well were removed, and each well was washed twice with 2 ml of PBS. Cells were then taken up with CellStripper (Thermo) reagent and transferred into 1.5 ml tubes. After pelleting and washing in PBS, cells were resuspended in 150 μl of PBS and counted. Defined numbers in 50 μl volumes in a 96-well Blackwell plate (Corning) were assayed for fluorescence in a fluorescent plate reader (Tecan).

Fluorescence arising from the reaction between DBCO-FAM and cell-surface azide label was also demonstrated by flow cytometry. In separate cultures, HeLa cells were added to wells of a 12-well plate at 10,000 cells/well, and cultured for 72 hours under normal conditions. The wells were washed with 1 ml of PBS, and 0.5 ml of DMEM-10% FBS was added. AzNAM was added to a final concentration of 125 μM, with cells receiving no azide-modified sugar as controls. After 20 hours, cells were harvested with CellStripper (250 μl/well), washed with PBS, and subjected to flow analysis.

By fluorescence readings in a 96-well plate format, it was demonstrated that cell-associated fluorescence increased as a function of the levels of AzNAM used in the initial labeling test. With flow analyses, marked and well-demarcated peak shifts were observed with the AzNAM-treated cells, which was corroborated by measuring fluorescence from equal numbers of cells in a fluorescent plate reader.

### Example 2: Labeling Cells Having Surface Azide-Modified Sugars with Bifunctional Compounds (Prophetic)

Cells that have been metabolically labeled with surface azide-modified sugars (see, Example 1) can be subsequently reacted with the bifunctional compounds described herein. After such reactions have occurred and excess compound is removed, the portion of the bifunctional molecule that is a small molecule ligand that binds to an FKBP domain or FRB domain can be displayed on the surface of the cell and can be available for subsequent reactions with a haplomer comprising an FKBP domain or FRB domain. Alternately, the portion of the bifunctional molecule that is a small molecule ligand that binds to an FKBP domain or FRB domain can first be allowed to react with the haplomer comprising an FKBP domain or FRB domain, after which the exposed azide reactive molecule of the bifunctional compound can be used for targeting the complex to the cell surface labeled with azide-modified sugars.

In particular, any of the bifunctional compounds described herein can be used for the purposes of cell surface positioning of any of the haplomers described herein. Cells displaying azide moieties on surface glycan molecules (as in Example 1) can be treated with 1 mM of the bifunctional compound (initially solubilized in DMSO as a 100 mM stock solution and diluted accordingly to the final desired concentration) in serum-free RPMI medium for 2 hours at room temperature in the presence of 1 mg/ml bovine serum albumin (BSA) (Sigma) and 500 μg/ml salmon sperm DNA. This treatment is followed by centrifugation (5 minutes at 2000 rpm in an Eppendorf centrifuge), followed by two washes with serum-free RPMI medium, with resuspension in 100 μl of the same medium. Following this, a haplomer comprising an FKBP domain or FRB domain can be added to the bifunctional compound-modified cells at a concentration of 1 pmol/μl, for a one hour incubation at room temperature. The cell preparations can be repelleted, washed twice with serum-free RPMI medium and once with PBS, with a final resuspension in 100 μl of PBS.

In an alternate embodiment, the haplomer comprising an FKBP domain or FRB domain can be pre-incubated with excess bifunctional compound prior to exposure to the target cells displaying surface azide. The haplomer in PBS (100 pmol) can be incubated with a 10-fold molar excess of the bifunctional compound for one hour at room temperature, followed by passage through a PBS-equilibrated P6 desalting column (Bio-Rad) to remove excess bifunctional compound. The resulting haplomer-bifunctional compound can be used to treat cells having surface azide, followed by washing steps as above.

### Example 3: Assaying Surface Glycan Density by Cis- Vs. Trans-Gaussia Split-Protein Complementation (Prophetic)

Cell surface template complementation of Gaussia luciferase split-protein (SP) fragments can be exploited as a distinct method for measurement of surface density of cell surface glycans that are metabolically modifiable with azides via provision of AzNAM. A single nuclease-resistant DBCO-labeled template nucleic acid molecule can be used, where it is separately hybridized with oligonucleotides complementary to both of its haplomer binding sites (see, FIG. 1).

The assay is performed by treating target cells bearing metabolically-placed surface azide groups (as described in Example 1) with DBCO-labeled templates in either a completely accessible state (see, FIG. 3) or as an equimolar mix of DBCO-templates with separately hybridization-masked sites for both haplomer binding sites (see, FIG. 4). After washing the cells twice with serum-free RPMI medium, Gaussia haplomers (see, FIG. 2, and PCT Publication WO 2018094195A1) targeting the template of interest are added in an equimolar ratio. The preparations of template-bearing cells and Gaussia haplomers are incubated for a further hour at room temperature, and washed once with serum-free RPMI, and once with PBS. Samples are then taken for measurement of luminescence generated by catalytically competent (refolded) Gaussia luciferase in a standard luminometer. The ratio of luminescent signal from open template vs. equimolar half-masked templates is indicative of the surface density of azide-modified glycans, where the magnitude of the open template:masked template ratio for Gaussia split-protein assembly is directly proportional to density.

### Example 4: Assaying Surface Glycan Density by Separate DBCO-Half-Templates (Prophetic)

In a variation of Example 3, instead of masking a single template, two separate DBCO-labeled half-template molecules are used. Cells bearing metabolically-placed surface azides (see, Examples 1 and 3) are separately treated with full-length open DBCO-template (see, FIG. 3), and equimolar mixtures of DBCO-labeled half-templates for both haplomer sites (see, FIG. 5). In a similar fashion to Example 3, Gaussia haplomers (see, FIGS. 2, and PCT Publication WO 2018094195A1) targeting the template of interest are added in an equimolar ratio. After suitable washes (see, Example 3), luminescence is determined, with the same read-out ratios relating to relative densities as for Example 3.

### Example 5: Split-Protein Complementation on Cell Surfaces Via Surface Azide and MFL4-DB (Prophetic)

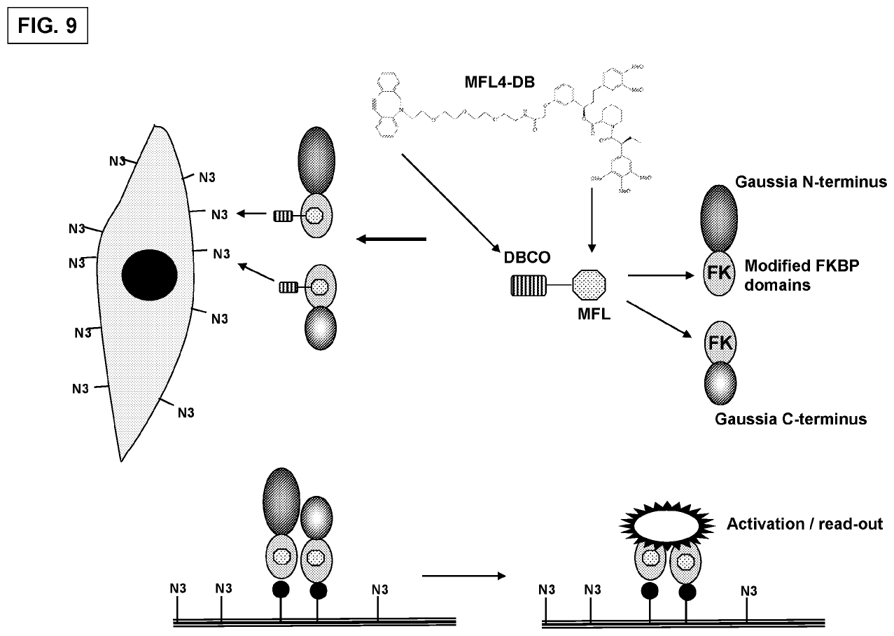

To create biologically active molecules on cell surfaces, split-protein fragments fused with the FKBP domain may be positioned on cell surfaces in a comparable manner to an intact protein of interest. Where equimolar mixtures of N- and C-terminal fragments are used for direct cell surface placement, the frequency of their juxtaposition which results in productive protein assembly will be a function of surface azide density (see, FIG. 9).

Gaussia split-protein fragments fused with a modified FKBP domain bearing the mutations C22V and F36V (see, PCT Publication WO 2018094195A1) are used. Samples of both the N-terminal and C-terminal Gaussia-FKBP fragments (100 pmol) are mixed with a 10-fold excess of MFL4-DB (see, FIG. 9) and incubated for 1 hour at room temperature. Excess MFL4-DB is removed by passage of the protein complexes though PBS-equilibrated P6 desalting columns (Bio-Rad). Binding of MFL4-DB in these complexes is mediated via the MFL (Monovalent FKBP Ligand) moiety of the compound, while the DBCO moiety is accessible to reaction with azides. AzNAM-treated HeLa cells (see, Example 1) are harvested, washed, and resuspended in serum-free RPMI to a concentration of 10⁶ cells/ml in 50 μl. Target cells are incubated with equimolar mixtures of N-terminal and C-terminal Gaussia-FKBP-MFL4-DB complexes (0.5 pmol/μl final concentration; see, FIG. 9) in the presence of 1 mg/ml of BSA (Sigma) and 500 μg/ml salmon sperm DNA, for 2 hours at room temperature, while ensuring that the cells are maintained in suspension. Control incubations performed in parallel include the use of: 1) HeLa cells grown under the same conditions as the AzNAM-treated cells, but without exposure to AzNAM; 2) Gaussia-FKBP fragments added in the same amounts as for the above MFL4-DB complexes but without addition of the compound; and 3) single additions of Gaussia N-terminal FKBP fragment fusion MFL4-DB complex and Gaussia C-terminal FKBP fragment fusion MFL4-DB complex, each applied separately to the target cells.

Following the incubation of cells and Gaussia fragments and complexes, the cells are pelleted, washed twice with serum-free RPMI and once with PBS, before final resuspension in 50 μl of PBS. Gaussia-derived luminescence in samples is assayed with 2 μl samples in a standard luminometer with coelenterazine substrate. Experimental success is determined by significant luminescence only in preparations where the HeLa cells display surface azide (as a consequence of prior AzNAM treatment), and only where both N- and C-terminal Gaussia FKBP fusions are added, and only where both are complexed with MFL4-DB.

Alternately, the methods for gauging surface glycan density can be determined by the extent of successful surface assembly with metabolically-placed azide groups. Here, the luminescent measurements with the above split-protein Gaussia are compared with luminescence obtained from corresponding target cells exposed to full-length Gaussia-FKBP fusion, where resulting surface Gaussia luciferase activity is only limited by the availability of surface azides. Since the frequency of productive assembly of surface split-protein fragments is strongly related to surface density of each bound fragment (see, FIG. 9), the ratio of (surface split-Gaussia luminescence)/(surface whole-Gaussia luminescence) provides a quantitative gauge for surface densities of the azide-modified glycans by which the process is carried out.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A method of determining surface glycan density of a cell, the method comprising the steps:
a) contacting the cell with an azide-modified sugar;
b) contacting the cell with a nucleic acid template comprising a 5′- or 3′-azide reactive molecule that is chemically reactable with an azide group, wherein the nucleic acid template has a first haplomer hybridization region and a second haplomer hybridization region;
c) contacting the cell with a first haplomer comprising:
i) a first oligonucleotide conjugated to a first ligand, wherein the first oligonucleotide is complementary to the first haplomer hybridization region; and
ii) a first fragment of a reporter molecule conjugated to a first ligand binding domain;
wherein the first ligand and first ligand binding domain associate with one another;
d) contacting the cell with a second haplomer comprising:
i) a second oligonucleotide conjugated to a second ligand, wherein the second oligonucleotide is complementary to the second haplomer hybridization region; and
ii) a second fragment of the reporter molecule conjugated to a second ligand binding domain;
wherein the second ligand and the second ligand binding domain associate with one another;
whereby, upon hybridization of the first oligonucleotide to the first haplomer hybridization region and the second oligonucleotide to the second haplomer hybridization region, and refolding of the first fragment of the reporter molecule with the second fragment of the reporter molecule, a functional reporter molecule is produced; and
e) detecting the amount of reporter molecule activity.

2. The method according to claim 1, wherein the azide reactive molecule is dibenzocyclooctyne (DBCO), bicyclo[6.1.0]nonyne (BCN), methyltetrazine, or trans-cyclooctene (TCO).

3. The method according to claim 1 or claim 2, wherein the azide-modified sugar is azido-N-acetylmannosamine (AzNAM), azido-N-acetylglucosamine (AzGlcNAc), azido-N-acetylgalactosamine (AGalNAc), or azido-N-acetylneuraminic acid (AzNANA).

4. The method according to any one of claims 1 to 3, wherein the azide-modified sugar is acetylated at 1, 2, 3, or 4 positions.

5. The method of any one of claims 1 to 4, wherein the nucleic acid template comprising the 5′- or 3′-azide reactive molecule is a mixture of two nucleic acid templates comprising the 5′- or 3′-azide reactive molecule, wherein the first nucleic acid template comprising the 5′- or 3′-azide reactive molecule is hybridized to a blocking oligonucleotide that is complementary to the first haplomer hybridization region, and wherein the second nucleic acid template comprising the 5′- or 3′-azide reactive molecule is hybridized to a blocking oligonucleotide that is complementary to the second haplomer hybridization region.

6. The method of any one of claims 1 to 5, wherein the first ligand and second ligand are small molecule ligands.

7. The method of claim 6, wherein the small molecule ligand is an FKBP-binding compound.

8. The method of claim 7, wherein the FKBP-binding compound is chosen from FKM-NHS, FKM-sulfo-NHS, FKM-PEG3-NHS, monovalent FKBP Ligand-2 (MFL2), FKM-PEG3-MTZ-NHS, and FKM-PEG3-TCO-NHS.

9. The method of any one of claims 1 to 8, wherein the first ligand binding domain and second ligand binding domains are FKBP domains or FRB domains.

10. The method of claim 9, wherein the FKBP domain is a mutant FKBP domain.

11. The method of claim 10, wherein the mutant FKBP domain is the F36V FKBP mutant domain comprising the amino acid sequence GVQVETISPGDGRTFPKRGQTCVVH YTGMLEDGKKVDSSRDRNKPFKFMLGKQEVIRGWEEGVAQMSVGQRAKLTISPDY AYGATGHPGIIPPHATLVFDVELLKLE (SEQ ID NO: 1) or MGVQVETISPGDGRTFPKR GQTCVVHYTGMLEDGKKVDSSRDRNKPFKFMLGKQEVIRGWEEGVAQMSVGQRA KLTISPDYAYGATGHPGIIPPHATLVFDVELLKLE (SEQ ID NO:2).

12. The method of claim 10, wherein the mutant FKBP domain comprises a C22S, C22A, or C22V substitution.

13. The method of claim 9, wherein the FRB domain comprises a C61S, C61A, or C61V substitution.

14. The method of any one of claims 1 to 5, wherein the first ligand and first ligand binding domain and/or the second ligand and second ligand binding domain are small interactive protein domain pairs.

15. The method of claim 14, wherein the small interactive protein domain pairs are chosen from jun/fos, mad/max, myc/max, and NZ/CZ domains.

16. The method according to any one of claims 1 to 15, wherein the reporter molecule is chosen from a luminescent protein, murine dihydrofolate reductase (DHFR), S. cerevisiae ubiquitin, β-lactamase, and Herpes simplex virus type 1 thymidine kinase.

17. The method according to claim 16, wherein the reporter molecule is DHFR, and one of the first fragment of the reporter molecule and the second fragment of the reporter molecule comprises amino acids 1-105 of DHFR, and the other of the first fragment of the reporter molecule and second fragment of the reporter molecule comprises amino acids 106-186 of DHFR.

18. The method according to claim 16, wherein the reporter molecule is S. cerevisiae ubiquitin, and one of the first fragment of the reporter molecule and the second fragment of the reporter molecule comprises amino acids 1-34 (MQIFVKTLTGKTITLEVESSDTIDNV KSKIQDKE; SEQ ID NO:3) of S. cerevisiae ubiquitin, and the other of the first fragment of the reporter molecule and second fragment of the reporter molecule comprises amino acids 35-76 (GIPPDQQRLIFAGKQLEDGRTLSDYNIQKESTLHLVLRLRGG; SEQ ID NO:4) of S. cerevisiae ubiquitin.

19. The method according to claim 16, wherein the reporter molecule is β-lactamase, and one of the first fragment of the reporter molecule and the second fragment of the reporter molecule comprises amino acids 25-197 of β-lactamase, and the other of the first fragment of the reporter molecule and second fragment of the reporter molecule comprises amino acids 198-286 of β-lactamase.

20. The method according to claim 16, wherein the reporter molecule is Herpes simplex virus type 1 thymidine kinase, and one of the first fragment of the reporter molecule and the second fragment of the reporter molecule comprises amino acids 1-265 of Herpes simplex virus type 1 thymidine kinase, and the other of the first fragment of the reporter molecule and second fragment of the reporter molecule comprises amino acids 266-376 of Herpes simplex virus type 1 thymidine kinase.

21. The method according to claim 16, wherein the luminescent protein is Gaussia luciferase, superfolder GFP (sfGFP), Renilla luciferase, or Nanoluc luciferase.

22. The method according to claim 21, wherein the luminescent protein is Gaussia luciferase, and one of the first fragment of Gaussia luciferase and second fragment of Gaussia luciferase comprises MKPTENNEDFNIVAVASNFATTDLDADRGKLPGKKLPLEVLKE MEANARKAGCTRGCLICLSHIKCTPKMKKFIPGRCHTYEGDKESAQGGIG (SEQ ID NO:5), and the other of the first fragment of Gaussia luciferase and second fragment of Gaussia luciferase comprises EAIVDIPEIPGFKDLEPMEQFIAQVDLCVDCTTGCLKGLA NVQCSDLLKKWLPQRCATFASKIQGQVDKIKGAGGD (SEQ ID NO:6).

23. The method according to claim 21, wherein the luminescent protein is sfGFP, and one of the first fragment of sfGFP and second fragment of sfGFP comprises MRKGEELFT GVVPILVELDGDVNGHKFSVRGEGEGDATNGKLTLKFICTTGKLPVPWPTLVTTLTY GVQCFARYPDHMKQHDFFKSAMPEGYVQERTISFKDDGTYKTRAEVKFEGDTLVN RIELKGIDFKEDGNILGHKLEYNFNSHNVYITADKQ (SEQ ID NO:7) or MSKGEELFT GVVPILVELDGDVNGHKFSVRGEGEGDATNGKLTLKFICTTGKLPVPWPTLVTTLTY GVQCFSRYPDHMKRHDFFKSAMPEGYVQERTISFKDDGTYKTRAEVKFEGDTLVNR IELKGIDFKEDGNILGHKLEYNFNSHNVYITADKQ (SEQ ID NO:22), and the other of the first fragment of sfGFP and second fragment of sfGFP comprises KNGIKANFKIRHNV EDGSVQLADHYQQNTPIGDGPVLLPDNHYLSTQSVLSKDPNEKRDHMVLLEFVTAA GITHGMDELYK (SEQ ID NO:8).

24. The method according to claim 21, wherein the luminescent protein is Renilla luciferase, and one of the first fragment of Renilla luciferase and second fragment of Renilla luciferase comprises MASKVYDPEQRKRMITGPQWWARCKQMNVLDSFINYYDSEKH AENAVIFLHGNAASSYLWRHVVPHIEPVARCIIPDLIGMGKSGKSGNGSYRLLDHYK YLTAWFELLNLPKKIIFVGHDWGACLAFHYSYEHQDKIKAIVHAESVVDVIESWDE WPDIEEDIALIKSEEGEKMVLENNFFVETMLPSKIMRKLEPEEFAAYLEPFKEKGEVR RPTLSWPREIPLVKGG (SEQ ID NO:9), and the other of the first fragment of Renilla luciferase and second fragment of Renilla luciferase comprises KPDVVQIVRNYNAYLRAS DDLPKMFIESDPGFFSNAIVEGAKKFPNTEFVKVKGLHFSQEDAPDEMGKYIKSFVER VLKNEQ (SEQ ID NO:10).

25. The method according to claim 21, wherein the luminescent protein is Nanoluc luciferase, and one of the first fragment of Nanoluc luciferase and second fragment of Nanoluc luciferase comprises MFTLEDFVGDWEQTAAYNLDQVLEQGGVSSLLQNLAV SVTPIQRIVRSGENALKIDIHVIIPYEGLSADQMAQIEEVFKVVYPVDDHHFKVILPYG TLVIDGVTPNMLNYFGRPYEGIAVFDGKKITVTGTLWNGNKIIDERLITPDGSMLFRV TINS (SEQ ID NO:34), and the other of the first fragment of Nanoluc luciferase and second fragment of Nanoluc luciferase comprises VSGWRLFKKIS (SEQ ID NO:35) or VTGYRL FEEIL (SEQ ID NO:36).

26. A method of determining surface glycan density of a cell, the method comprising the steps:
a) contacting the cell with an azide-modified sugar;
b) contacting the cell with a first nucleic acid template comprising a 5′-azide reactive molecule that is chemically reactable with an azide group, wherein the nucleic acid template has a first haplomer hybridization region;
c) contacting the cell with a second nucleic acid template comprising a 3′-azide reactive molecule that is chemically reactable with an azide group, wherein the nucleic acid template has a second haplomer hybridization region;
d) contacting the cell with a first haplomer comprising:
i) a first oligonucleotide conjugated to a first ligand, wherein the first oligonucleotide is complementary to the first haplomer hybridization region; and
ii) a first fragment of a reporter molecule conjugated to a first ligand binding domain;
wherein the first ligand and the first ligand binding domain associate with one another;
e) contacting the cell with a second haplomer comprising:
i) a second oligonucleotide conjugated to a second ligand, wherein the second oligonucleotide is complementary to the second haplomer hybridization region; and
ii) a second fragment of the reporter molecule conjugated to a second ligand binding domain;
wherein the second ligand and the second ligand binding domain associate with one another;
whereby, upon hybridization of the first oligonucleotide to the first haplomer hybridization region and the second oligonucleotide to the second haplomer hybridization region, and refolding of the first fragment of the reporter molecule with the second fragment of the reporter molecule, a functional reporter molecule is produced; and
f) detecting the amount of reporter molecule activity.

27. The method according to claim 26, wherein the azide reactive molecule is dibenzocyclooctyne (DBCO), bicyclo[6.1.0]nonyne (BCN), methyltetrazine, or trans-cyclooctene (TCO).

28. The method according to claim 26 or claim 27, wherein the azide-modified sugar is azido-N-acetylmannosamine (AzNAM), azido-N-acetylglucosamine (AzGlcNAc), azido-N-acetylgalactosamine (AGalNAc), or azido-N-acetylneuraminic acid (AzNANA).

29. The method according to any one of claims 26 to 28, wherein the azide-modified sugar is acetylated at 1, 2, 3, or 4 positions.

30. The method of any one of claims 26 to 29, wherein the first ligand and second ligand are small molecule ligands.

31. The method of claim 30, wherein the small molecule ligand is an FKBP-binding compound.

32. The method of claim 31, wherein the FKBP-binding compound is chosen from FKM-NHS, FKM-sulfo-NHS, FKM-PEG3-NHS, monovalent FKBP Ligand-2 (MFL2), FKM-PEG3-MTZ-NHS, and FKM-PEG3-TCO-NHS.

33. The method of any one of claims 26 to 32, wherein the first ligand binding domain and second ligand binding domains are FKBP domains or FRB domains.

34. The method of claim 33, wherein the FKBP domain is a mutant FKBP domain.

35. The method of claim 34, wherein the mutant FKBP domain is the F36V FKBP mutant domain comprising the amino acid sequence GVQVETISPGDGRTFPKRGQTCVVH YTGMLEDGKKVDSSRDRNKPFKFMLGKQEVIRGWEEGVAQMSVGQRAKLTISPDY AYGATGHPGIIPPHATLVFDVELLKLE (SEQ ID NO: 1) or MGVQVETISPGDGRTFPKR GQTCVVHYTGMLEDGKKVDSSRDRNKPFKFMLGKQEVIRGWEEGVAQMSVGQRA KLTISPDYAYGATGHPGIIPPHATLVFDVELLKLE (SEQ ID NO:2).

36. The method of claim 34, wherein the mutant FKBP domain comprises a C22S, C22A, or C22V substitution.

37. The method of claim 33, wherein the FRB domain comprises a C61S, C61A, or C61V substitution.

38. The method of any one of claims 26 to 29, wherein the first ligand and first ligand binding domain and/or the second ligand and second ligand binding domain are small interactive protein domain pairs.

39. The method of claim 38, wherein the small interactive protein domain pairs are chosen from jun/fos, mad/max, myc/max, and NZ/CZ domains.

40. The method according to any one of claims 26 to 39, wherein the reporter molecule is chosen from a luminescent protein, murine dihydrofolate reductase (DHFR), S. cerevisiae ubiquitin, β-lactamase, and Herpes simplex virus type 1 thymidine kinase.

41. The method according to claim 40, wherein the reporter molecule is DHFR, and one of the first fragment of the reporter molecule and the second fragment of the reporter molecule comprises amino acids 1-105 of DHFR, and the other of the first fragment of the reporter molecule and second fragment of the reporter molecule comprises amino acids 106-186 of DHFR.

42. The method according to claim 40, wherein the reporter molecule is S. cerevisiae ubiquitin, and one of the first fragment of the reporter molecule and the second fragment of the reporter molecule comprises amino acids 1-34 (MQIFVKTLTGKTITLEVESSDTIDNV KSKIQDKE; SEQ ID NO:3) of S. cerevisiae ubiquitin, and the other of the first fragment of the reporter molecule and second fragment of the reporter molecule comprises amino acids 35-76 (GIPPDQQRLIFAGKQLEDGRTLSDYNIQKESTLHLVLRLRGG; SEQ ID NO:4) of S. cerevisiae ubiquitin.

43. The method according to claim 40, wherein the reporter molecule is β-lactamase, and one of the first fragment of the reporter molecule and the second fragment of the reporter molecule comprises amino acids 25-197 of β-lactamase, and the other of the first fragment of the reporter molecule and second fragment of the reporter molecule comprises amino acids 198-286 of β-lactamase.

44. The method according to claim 40, wherein the reporter molecule is Herpes simplex virus type 1 thymidine kinase, and one of the first fragment of the reporter molecule and the second fragment of the reporter molecule comprises amino acids 1-265 of Herpes simplex virus type 1 thymidine kinase, and the other of the first fragment of the reporter molecule and second fragment of the reporter molecule comprises amino acids 266-376 of Herpes simplex virus type 1 thymidine kinase.

45. The method according to claim 40, wherein the luminescent protein is Gaussia luciferase, superfolder GFP (sfGFP), Renilla luciferase, or Nanoluc luciferase.

46. The method according to claim 45, wherein the luminescent protein is Gaussia luciferase, and one of the first fragment of Gaussia luciferase and second fragment of Gaussia luciferase comprises MKPTENNEDFNIVAVASNFATTDLDADRGKLPGKKLPLEVLKE MEANARKAGCTRGCLICLSHIKCTPKMKKFIPGRCHTYEGDKESAQGGIG (SEQ ID NO:5), and the other of the first fragment of Gaussia luciferase and second fragment of Gaussia luciferase comprises EAIVDIPEIPGFKDLEPMEQFIAQVDLCVDCTTGCLKGLA NVQCSDLLKKWLPQRCATFASKIQGQVDKIKGAGGD (SEQ ID NO:6).

47. The method according to claim 45, wherein the luminescent protein is sfGFP, and one of the first fragment of sfGFP and second fragment of sfGFP comprises MRKGEELFTG VVPILVELDGDVNGHKFSVRGEGEGDATNGKLTLKFICTTGKLPVPWPTLVTTLTYG VQCFARYPDHMKQHDFFKSAMPEGYVQERTISFKDDGTYKTRAEVKFEGDTLVNRI ELKGIDFKEDGNILGHKLEYNFNSHNVYITADKQ (SEQ ID NO:7) or MSKGEELFTGV VPILVELDGDVNGHKFSVRGEGEGDATNGKLTLKFICTTGKLPVPWPTLVTTLTYGV QCFSRYPDHMKRHDFFKSAMPEGYVQERTISFKDDGTYKTRAEVKFEGDTLVNRIEL KGIDFKEDGNILGHKLEYNFNSHNVYITADKQ (SEQ ID NO:22), and the other of the first fragment of sfGFP and second fragment of sfGFP comprises KNGIKANFKIRHNVEDG SVQLADHYQQNTPIGDGPVLLPDNHYLSTQSVLSKDPNEKRDHMVLLEFVTAAGITH GMDELYK (SEQ ID NO:8).

48. The method according to claim 45, wherein the luminescent protein is Renilla luciferase, and one of the first fragment of Renilla luciferase and second fragment of Renilla luciferase comprises MASKVYDPEQRKRMITGPQWWARCKQMNVLDSFINYYDSEKH AENAVIFLHGNAASSYLWRHVVPHIEPVARCIIPDLIGMGKSGKSGNGSYRLLDHYK YLTAWFELLNLPKKIIFVGHDWGACLAFHYSYEHQDKIKAIVHAESVVDVIESWDE WPDIEEDIALIKSEEGEKMVLENNFFVETMLPSKIMRKLEPEEFAAYLEPFKEKGEVR RPTLSWPREIPLVKGG (SEQ ID NO:9), and the other of the first fragment of Renilla luciferase and second fragment of Renilla luciferase comprises KPDVVQIVRNYNAYLRAS DDLPKMFIESDPGFFSNAIVEGAKKFPNTEFVKVKGLHFSQEDAPDEMGKYIKSFVER VLKNEQ (SEQ ID NO:10).

49. The method according to claim 45, wherein the luminescent protein is Nanoluc luciferase, and one of the first fragment of Nanoluc luciferase and second fragment of Nanoluc luciferase comprises MFTLEDFVGDWEQTAAYNLDQVLEQGGVSSLLQNLAV SVTPIQRIVRSGENALKIDIHVIIPYEGLSADQMAQIEEVFKVVYPVDDHHFKVILPYG TLVIDGVTPNMLNYFGRPYEGIAVFDGKKITVTGTLWNGNKIIDERLITPDGSMLFRV TINS (SEQ ID NO:34), and the other of the first fragment of Nanoluc luciferase and second fragment of Nanoluc luciferase comprises VSGWRLFKKIS (SEQ ID NO:35) or VTGYRL FEEIL (SEQ ID NO:36).

50. A method of determining surface glycan density of a cell, the method comprising the steps:
a) contacting the cell with an azide-modified sugar;
b) contacting the cell with a bifunctional compound, wherein the bifunctional compound comprises an azide reactive molecule that is chemically reactable with an azide group, and a small molecule ligand that binds to an FKBP domain or FRB domain, wherein the bifunctional compound associates with a first glycan molecule, and another bifunctional compound associates with a second glycan molecule;
c) contacting the cell with a first haplomer comprising an FKBP domain or FRB domain conjugated to a first fragment of a reporter molecule;
d) contacting the cell with a second haplomer comprising an FKBP domain or FRB domain conjugated to a second fragment of the reporter molecule;
whereby, upon association between the FKBP domain or FRB domain of the first haplomer and the small molecule ligand of the bifunctional molecule of a first glycan molecule and the association between the FKBP domain or FRB domain of the second haplomer and the small molecule ligand of the bifunctional molecule of a second glycan molecule in sufficient proximity to the first glycan molecule, and refolding of the first fragment of the reporter molecule with the second fragment of the reporter molecule, a functional reporter molecule is produced; and
e) detecting the amount of reporter molecule activity.

51. The method according to claim 50, wherein the azide reactive molecule is dibenzocyclooctyne (DBCO), bicyclo[6.1.0]nonyne (BCN), methyltetrazine, or trans-cyclooctene (TCO).

52. The method according to claim 50 or claim 51, wherein the azide-modified sugar is azido-N-acetylmannosamine (AzNAM), azido-N-acetylglucosamine (AzGlcNAc), azido-N-acetylgalactosamine (AGalNAc), or azido-N-acetylneuraminic acid (AzNANA).

53. The method according to any one of claims 50 to 52, wherein the azide-modified sugar is acetylated at 1, 2, 3, or 4 positions.

54. The method of any one of claims 50 to 53, wherein the small molecule ligand is an FKBP-binding compound.

55. The method of claim 54, wherein the FKBP-binding compound is chosen from FKM-NHS, FKM-sulfo-NHS, FKM-PEG3-NHS, monovalent FKBP Ligand-2 (MFL2), FKM-PEG3-MTZ-NHS, and FKM-PEG3-TCO-NHS.

56. The method of any one of claims 50 to 55, wherein the FKBP domain is a mutant FKBP domain.

57. The method of claim 56, wherein the mutant FKBP domain is the F36V FKBP mutant domain comprising the amino acid sequence GVQVETISPGDGRTFPKRGQTCVV HYTGMLEDGKKVDSSRDRNKPFKFMLGKQEVIRGWEEGVAQMSVGQRAKLTISPD YAYGATGHPGIIPPHATLVFDVELLKLE (SEQ ID NO: 1) or MGVQVETISPGDGRTFPK RGQTCVVHYTGMLEDGKKVDSSRDRNKPFKFMLGKQEVIRGWEEGVAQMSVGQR AKLTISPDYAYGATGHPGIIPPHATLVFDVELLKLE (SEQ ID NO:2).

58. The method of claim 56, wherein the mutant FKBP domain comprises a C22S, C22A, or C22V substitution.

59. The method of any one of claims 50 to 55, wherein the FRB domain comprises a C61S, C61A, or C61V substitution.

60. The method according to any one of claims 50 to 59, wherein the reporter molecule is chosen from a luminescent protein, murine dihydrofolate reductase (DHFR), S. cerevisiae ubiquitin, β-lactamase, and Herpes simplex virus type 1 thymidine kinase.

61. The method according to claim 60, wherein the reporter molecule is DHFR, and one of the first fragment of the reporter molecule and the second fragment of the reporter molecule comprises amino acids 1-105 of DHFR, and the other of the first fragment of the reporter molecule and second fragment of the reporter molecule comprises amino acids 106-186 of DHFR.

62. The method according to claim 60, wherein the reporter molecule is S. cerevisiae ubiquitin, and one of the first fragment of the reporter molecule and the second fragment of the reporter molecule comprises amino acids 1-34 (MQIFVKTLTGKTITLEVESSDTIDNV KSKIQDKE; SEQ ID NO:3) of S. cerevisiae ubiquitin, and the other of the first fragment of the reporter molecule and second fragment of the reporter molecule comprises amino acids 35-76 (GIPPDQQRLIFAGKQLEDGRTLSDYNIQKESTLHLVLRLRGG; SEQ ID NO:4) of S. cerevisiae ubiquitin.

63. The method according to claim 60, wherein the reporter molecule is β-lactamase, and one of the first fragment of the reporter molecule and the second fragment of the reporter molecule comprises amino acids 25-197 of β-lactamase, and the other of the first fragment of the reporter molecule and second fragment of the reporter molecule comprises amino acids 198-286 of β-lactamase.

64. The method according to claim 60, wherein the reporter molecule is Herpes simplex virus type 1 thymidine kinase, and one of the first fragment of the reporter molecule and the second fragment of the reporter molecule comprises amino acids 1-265 of Herpes simplex virus type 1 thymidine kinase, and the other of the first fragment of the reporter molecule and second fragment of the reporter molecule comprises amino acids 266-376 of Herpes simplex virus type 1 thymidine kinase.

65. The method according to claim 60, wherein the luminescent protein is Gaussia luciferase, superfolder GFP (sfGFP), Renilla luciferase, or Nanoluc luciferase.

66. The method according to claim 65, wherein the luminescent protein is Gaussia luciferase, and one of the first fragment of Gaussia luciferase and second fragment of Gaussia luciferase comprises MKPTENNEDFNIVAVASNFATTDLDADRGKLPGKKLPLEVLKE MEANARKAGCTRGCLICLSHIKCTPKMKKFIPGRCHTYEGDKESAQGGIG (SEQ ID NO:5), and the other of the first fragment of Gaussia luciferase and second fragment of Gaussia luciferase comprises EAIVDIPEIPGFKDLEPMEQFIAQVDLCVDCTTGCLKGLA NVQCSDLLKKWLPQRCATFASKIQGQVDKIKGAGGD (SEQ ID NO:6).

67. The method according to claim 65, wherein the luminescent protein is sfGFP, and one of the first fragment of sfGFP and second fragment of sfGFP comprises MRKGEELFTG VVPILVELDGDVNGHKFSVRGEGEGDATNGKLTLKFICTTGKLPVPWPTLVTTLTYG VQCFARYPDHMKQHDFFKSAMPEGYVQERTISFKDDGTYKTRAEVKFEGDTLVNRI ELKGIDFKEDGNILGHKLEYNFNSHNVYITADKQ (SEQ ID NO:7) or MSKGEELFTGV VPILVELDGDVNGHKFSVRGEGEGDATNGKLTLKFICTTGKLPVPWPTLVTTLTYGV QCFSRYPDHMKRHDFFKSAMPEGYVQERTISFKDDGTYKTRAEVKFEGDTLVNRIEL KGIDFKEDGNILGHKLEYNFNSHNVYITADKQ (SEQ ID NO:22), and the other of the first fragment of sfGFP and second fragment of sfGFP comprises KNGIKANFKIRHNVEDG SVQLADHYQQNTPIGDGPVLLPDNHYLSTQSVLSKDPNEKRDHMVLLEFVTAAGITH GMDELYK (SEQ ID NO:8).

68. The method according to claim 65, wherein the luminescent protein is Renilla luciferase, and one of the first fragment of Renilla luciferase and second fragment of Renilla luciferase comprises MASKVYDPEQRKRMITGPQWWARCKQMNVLDSFINYYDSEKH AENAVIFLHGNAASSYLWRHVVPHIEPVARCIIPDLIGMGKSGKSGNGSYRLLDHYK YLTAWFELLNLPKKIIFVGHDWGACLAFHYSYEHQDKIKAIVHAESVVDVIESWDE WPDIEEDIALIKSEEGEKMVLENNFFVETMLPSKIMRKLEPEEFAAYLEPFKEKGEVR RPTLSWPREIPLVKGG (SEQ ID NO:9), and the other of the first fragment of Renilla luciferase and second fragment of Renilla luciferase comprises KPDVVQIVRNYNAYLRAS DDLPKMFIESDPGFFSNAIVEGAKKFPNTEFVKVKGLHFSQEDAPDEMGKYIKSFVER VLKNEQ (SEQ ID NO:10).

69. The method according to claim 65, wherein the luminescent protein is Nanoluc luciferase, and one of the first fragment of Nanoluc luciferase and second fragment of Nanoluc luciferase comprises MFTLEDFVGDWEQTAAYNLDQVLEQGGVSSLLQNLAV SVTPIQRIVRSGENALKIDIHVIIPYEGLSADQMAQIEEVFKVVYPVDDHHFKVILPYG TLVIDGVTPNMLNYFGRPYEGIAVFDGKKITVTGTLWNGNKIIDERLITPDGSMLFRV TINS (SEQ ID NO:34), and the other of the first fragment of Nanoluc luciferase and second fragment of Nanoluc luciferase comprises VSGWRLFKKIS (SEQ ID NO:35) or VTGYRL FEEIL (SEQ ID NO:36).

70. The method according to any one of claims 50 to 69, wherein the bifunctional compound comprises the formula: 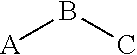
wherein:
A is a small molecule ligand that binds to an FKBP domain or FRB domain;
B is a chemical linker chosen from an alkyl, an alkenyl, an amide, an ester, a thioester, a ketone, an ether, a thioether, a disulfide, an ethylene glycol unit, a cycloalkyl, a benzyl, a heterocyclic, a maleimidyl, a hydrazone, a urethane, an azole, an imine, a haloalkyl, or a carbamate, or any combination thereof; and
C is an azide reactive molecule chosen from a cyclooctyne, a norbornene, an oxanorbornadiene, a phosphine, a dialkyl phosphine, a trialkyl phosphine, a phosphinothiol, a phosphinophenol, a cyclooctene, a tetrazine, a tetrazole, or a quadricyclane.

71. The method according to claim 70, wherein the FKBP domain is the FK506-FKBP domain or the mutant (F36V) FKBP domain.

72. The method according to claim 70 or claim 71, wherein the FKBP domain is the FK506-FKBP domain.

73. The method according to claim 70 or claim 71, wherein the FKBP domain is the mutant (F36V) FKBP domain.

74. The method according to claim 70 or claim 71, wherein the small molecule ligand is

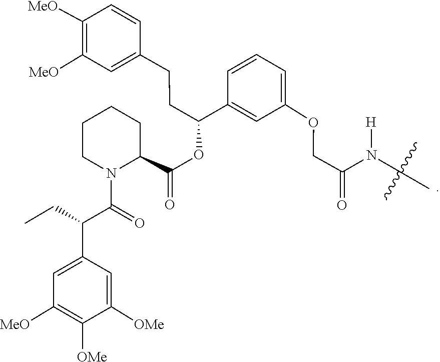 [SMILES: *NC(=O)COc1cccc([C@@H](CCc2ccc(OC)c(OC)c2)OC(=O)[C@@H]2CCCCN2C(=O)[C@@H](CC)c2cc(OC)c(OC)c(OC)c2)c1]

75. The method according to any one of claims 70 to 74, wherein the chemical linker is an alkyl or an ethylene glycol unit.

76. The method according to claim 75, wherein the chemical linker is an alkyl.

77. The method according to claim 76, wherein the chemical linker is a C₂-C₁₆alkyl.

78. The method according to claim 77, wherein the chemical linker is a C₄-C₁₂alkyl or a C₄-C₁₆alkyl.

79. The method according to claim 78, wherein the chemical linker is a C₄-C₁₀alkyl.

80. The method according to claim 79, wherein the chemical linker is C₄alkyl or C₁₀alkyl.

81. The method according to claim 75, wherein the chemical linker is an ethylene glycol unit.

82. The method according to claim 81, wherein the chemical linker is a polyethylene glycol (PEG).

83. The method according to claim 82, wherein the PEG is PEG2 to PEG16.

84. The method according to claim 83, wherein the PEG is PEG2, PEG3, or PEG4.

85. The method according to any one of claims 70 to 84, wherein the azide reactive molecule is chosen from a cyclooctyne, a cyclooctene, and a tetrazine.

86. The method according to claim 85, wherein the cyclooctyne is dibenzocyclooctyne (DBCO), bicyclo[6.1.0]nonyne (BCN), monofluorinated cyclooctyne, difluorocyclooctyne, dimethoxyazacyclooctyne, dibenzoazacyclooctyne, biarylazacyclooctynone, 2,3,6,7-tetramethoxy-dibenzocyclooctyne, sulfonylated dibenzocyclooctyne, carboxymethylmonobenzocyclooctyne, or pyrrolocyclooctyne.

87. The method according to claim 85, wherein the cyclooctene is trans-cyclooctene

88. The method according to claim 85, wherein the tetrazine is methyltetrazine, diphenyltetrazine, 3,6-di-(2-pyridyl)-s-tetrazine, 3,6-diphenyl-s-tetrazine, 3-(5-aminopyridin-2-yl)-6-(pyridin-2-yl)-s-tetrazine, or N-benzoyl-3-(5-aminopyridin-2-yl)-6-(pyridin-2-yl)-s-tetrazine.

89. The method according to claim 70, wherein:
the FKBP domain is the FK506-FKBP domain or the mutant (F36V) FKBP domain;
the chemical linker is an alkyl or an ethylene glycol unit; and
the azide reactive molecule is chosen from a cyclooctyne, a cyclooctene, and a tetrazine.

90. The method according to claim 70, wherein:
the FKBP domain is the FK506-FKBP domain or the mutant (F36V) FKBP domain;
the chemical linker is a C₂-C₁₆alkyl, or a polyethylene glycol which is PEG2 to PEG16; and
the azide reactive molecule is DBCO, BCN, TCO, or methyltetrazine.

91. The method according to claim 70, wherein:
the FKBP domain is the mutant (F36V), FKBP domain;
the chemical linker is a C₄-C₁₀alkyl or a polyethylene glycol which is PEG2, PEG3, or PEG4; and
the azide reactive molecule is DBCO, BCN, TCO, or methyltetrazine.

92. The method according to claim 70, wherein:
the small molecule ligand is 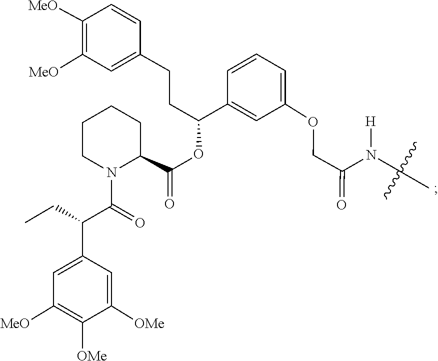 [SMILES: *NC(=O)COc1cccc([C@@H](CCc2ccc(OC)c(OC)c2)OC(=O)[C@@H]2CCCCN2C(=O)[C@@H](CC)c2cc(OC)c(OC)c(OC)c2)c1]
the chemical linker is C₄alkyl, C₁₀alkyl, or PEG3; and
the azide reactive molecule is DBCO or BCN.

93. The method according to claim 70, wherein the bifunctional compound comprises the formula:

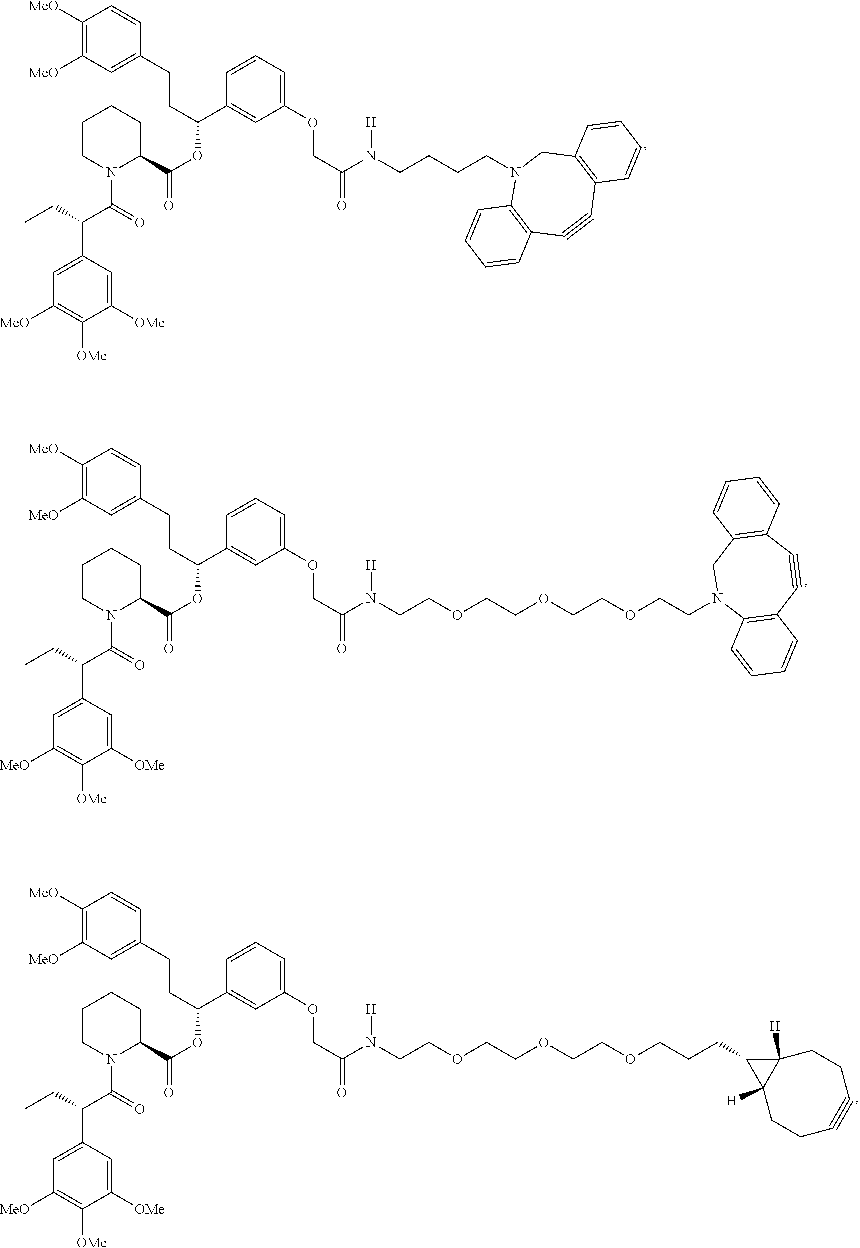 [SMILES: CC[C@H](C(=O)N1CCCC[C@H]1C(=O)O[C@H](CCc1ccc(OC)c(OC)c1)c1cccc(OCC(=O)NCCCCN2Cc3ccccc3C#Cc3ccccc32)c1)c1cc(OC)c(OC)c(OC)c1 ; CC[C@H](C(=O)N1CCCC[C@H]1C(=O)O[C@H](CCc1ccc(OC)c(OC)c1)c1cccc(OCC(=O)NCCOCCOCCOCCN2Cc3ccccc3C#Cc3ccccc32)c1)c1cc(OC)c(OC)c(OC)c1 ; CC[C@H](C(=O)N1CCCC[C@H]1C(=O)O[C@H](CCc1ccc(OC)c(OC)c1)c1cccc(OCC(=O)NCCOCCOCCOCCC[C@@H]2[C@@H]3CCC#CCC[C@@H]32)c1)c1cc(OC)c(OC)c(OC)c1]

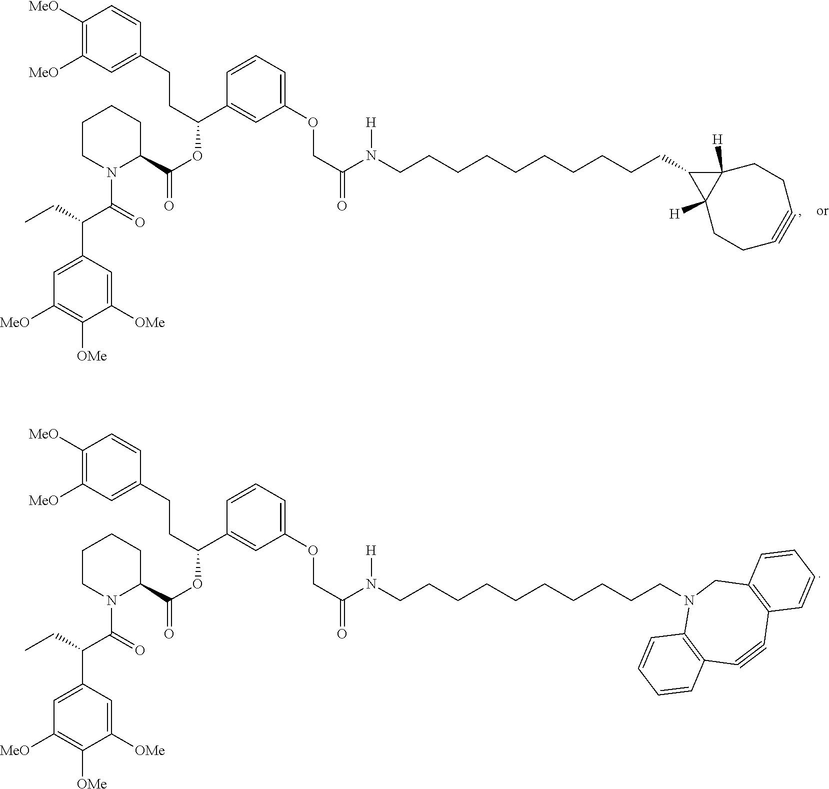 [SMILES: CC[C@H](C(=O)N1CCCC[C@H]1C(=O)O[C@H](CCc1ccc(OC)c(OC)c1)c1cccc(OCC(=O)NCCCCCCCCCC[C@@H]2[C@@H]3CCC#CCC[C@@H]32)c1)c1cc(OC)c(OC)c(OC)c1 ; CC[C@H](C(=O)N1CCCC[C@H]1C(=O)O[C@H](CCc1ccc(OC)c(OC)c1)c1cccc(OCC(=O)NCCCCCCCCCCN2Cc3ccccc3C#Cc3ccccc32)c1)c1cc(OC)c(OC)c(OC)c1]

94. The method according to any one of claims 50 to 93, wherein the first haplomer and second haplomer are pre-incubated with excess bifunctional compound prior to their contacting the cells.


SEQUENCE LISTING (40 sequences)

SEQ ID NO: 1 - protein, 107 aa, Artificial Sequence
  Description of Artificial Sequence Synthetic polypeptide
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 2 - protein, 108 aa, Artificial Sequence
  Description of Artificial Sequence Synthetic polypeptide
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 3 - protein, 34 aa, Saccharomyces cerevisiae
MQIFVKTLTGKTITLEVESSDTIDNVKSKIQDKE

SEQ ID NO: 4 - protein, 42 aa, Saccharomyces cerevisiae
GIPPDQQRLIFAGKQLEDGRTLSDYNIQKESTLHLVLRLRGG

SEQ ID NO: 5 - protein, 93 aa, Gaussia princeps
MKPTENNEDFNIVAVASNFATTDLDADRGKLPGKKLPLEVLKEMEANARKAGCTRGCLICLSHIKCTPKMKKFIPGRCHTYEGDKESAQGGIG

SEQ ID NO: 6 - protein, 76 aa, Gaussia princeps
EAIVDIPEIPGFKDLEPMEQFIAQVDLCVDCTTGCLKGLANVQCSDLLKKWLPQRCATFASKIQGQVDKIKGAGGD

SEQ ID NO: 7 - protein, 157 aa, Unknown
  Description of Unknown sfGFP sequence
MRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQ

SEQ ID NO: 8 - protein, 81 aa, Unknown
  Description of Unknown sfGFP sequence
KNGIKANFKIRHNVEDGSVQLADHYQQNTPIGDGPVLLPDNHYLSTQSVLSKDPNEKRDHMVLLEFVTAAGITHGMDELYK

SEQ ID NO: 9 - protein, 229 aa, Renilla reniformis
MASKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGG

SEQ ID NO: 10 - protein, 82 aa, Renilla reniformis
KPDVVQIVRNYNAYLRASDDLPKMFIESDPGFFSNAIVEGAKKFPNTEFVKVKGLHFSQEDAPDEMGKYIKSFVERVLKNEQ

SEQ ID NO: 11 - protein, 47 aa, Artificial Sequence
  Description of Artificial Sequence Synthetic polypeptide
CSGGASLERIARLEEKVKTLKAQNSELASTANMLREQVAQLKQKGAP

SEQ ID NO: 12 - protein, 49 aa, Artificial Sequence
  Description of Artificial Sequence Synthetic polypeptide
SGASLERIARLEEKVKTLKAQNSELASTANMLREQVAQLKQKGAPSGGC

SEQ ID NO: 13 - protein, 47 aa, Artificial Sequence
  Description of Artificial Sequence Synthetic polypeptide
CSGGASLERIARLEEKVKSFKAQNSENASTANMLREQVAQLKQKGAP

SEQ ID NO: 14 - protein, 49 aa, Artificial Sequence
  Description of Artificial Sequence Synthetic polypeptide
SGASLERIARLEEKVKSFKAQNSENASTANMLREQVAQLKQKGAPSGGC

SEQ ID NO: 15 - protein, 46 aa, Artificial Sequence
  Description of Artificial Sequence Synthetic polypeptide
CSGASLERIARLEEKVKSFKAQNSENASTANMLREQVAQLKQKGAP

SEQ ID NO: 16 - protein, 48 aa, Artificial Sequence
  Description of Artificial Sequence Synthetic polypeptide
GASLERIARLEEKVKTLKAQNSELASTANMLREQVAQLKQKGAPSGGC

SEQ ID NO: 17 - protein, 41 aa, Artificial Sequence
  Description of Artificial Sequence Synthetic polypeptide
ASRELTDTLQAETDQLEDEKSALQTEIANLLKEKEKLEGAP

SEQ ID NO: 18 - protein, 40 aa, Artificial Sequence
  Description of Artificial Sequence Synthetic polypeptide
ASRETDTLQAETDQLEDEKSALQTEIANLLKEKEKLEGAP

SEQ ID NO: 19 - protein, 43 aa, Artificial Sequence
  Description of Artificial Sequence Synthetic polypeptide
SGASRELTDTLQAETDQLEDEKSALQTEIANLLKEKEKLEGAP

SEQ ID NO: 20 - protein, 29 aa, Artificial Sequence
  Description of Artificial Sequence Synthetic peptide
ALKKELQANKKELAQLKWELQALKKELAQ

SEQ ID NO: 21 - protein, 30 aa, Artificial Sequence
  Description of Artificial Sequence Synthetic polypeptide
EQLEKKLQALEKKLAQLEWKNQALEKKLAQ

SEQ ID NO: 22 - protein, 157 aa, Unknown
  Description of Unknown sfGFP sequence
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQ

SEQ ID NO: 23 - protein, 10 aa, Artificial Sequence
  Description of Artificial Sequence Synthetic peptide
AGSSAAGSGS

SEQ ID NO: 24 - protein, 18 aa, Artificial Sequence
  Description of Artificial Sequence Synthetic peptide
GGSGGGSGGGSGGGSGGG

SEQ ID NO: 25 - protein, 22 aa, Artificial Sequence
  Description of Artificial Sequence Synthetic peptide
GGSGGGSGGGSGGGSGGGSGGG

SEQ ID NO: 26 - protein, 26 aa, Artificial Sequence
  Description of Artificial Sequence Synthetic peptide
GGSGGGSGGGSGGGSGGGSGGGSGGG

SEQ ID NO: 27 - protein, 15 aa, Artificial Sequence
  Description of Artificial Sequence Synthetic peptide
SGGGGSGGGGSGGGG

SEQ ID NO: 28 - protein, 20 aa, Artificial Sequence
  Description of Artificial Sequence Synthetic peptide
SGGGGSGGGGSGGGGSGGGG

SEQ ID NO: 29 - protein, 25 aa, Artificial Sequence
  Description of Artificial Sequence Synthetic peptide
SGGGGSGGGGSGGGGSGGGGSGGGG

SEQ ID NO: 30 - protein, 5 aa, Artificial Sequence
  Description of Artificial Sequence Synthetic peptide
SGGGS

SEQ ID NO: 31 - protein, 4 aa, Artificial Sequence
  Description of Artificial Sequence Synthetic peptide
SGSG

SEQ ID NO: 32 - protein, 6 aa, Artificial Sequence
  Description of Artificial Sequence Synthetic peptide
SGGGGS

SEQ ID NO: 33 - protein, 5 aa, Artificial Sequence
  Description of Artificial Sequence Synthetic peptide
SGSGG

SEQ ID NO: 34 - protein, 158 aa, Oplophorus gracilirostris
MFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 35 - protein, 11 aa, Oplophorus gracilirostris
VSGWRLFKKIS

SEQ ID NO: 36 - protein, 11 aa, Oplophorus gracilirostris
VTGYRLFEEIL

SEQ ID NO: 37 - protein, 302 aa, Artificial Sequence
  Description of Artificial Sequence Synthetic polypeptide
MFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINGVTGWRLCERILASGGGGSGGGGSGGGSGVQVETISPGDGRTFPKRGQTVVVHYTGMLEDGKKVDSSRDRNKPFKFMLGKQEVIRGWEEGVAQMSVGQRAKLTISPDYAYGATGHPGIIPPHATLVFDVELLKLEGGSGHHHHHH

SEQ ID NO: 38 - protein, 289 aa, Artificial Sequence
  Description of Artificial Sequence Synthetic polypeptide
MFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGGGSGGGGSGGGSGVQVETISPGDGRTFPKRGQTVVVHYTGMLEDGKKVDSSRDRNKPFKFMLGKQEVIRGWEEGVAQMSVGQRAKLTISPDYAYGATGHPGIIPPHATLVFDVELLKLEGGSGHHHHHH

SEQ ID NO: 39 - protein, 144 aa, Artificial Sequence
  Description of Artificial Sequence Synthetic polypeptide
MHHHHHHGGSGGVQVETISPGDGRTFPKRGQTVVVHYTGMLEDGKKVDSSRDRNKPFKFMLGKQEVIRGWEEGVAQMSVGQRAKLTISPDYAYGATGHPGIIPPHATLVFDVELLKLESGGGGSGGGGSGGGSVSGWRLFKKIS

SEQ ID NO: 40 - protein, 144 aa, Artificial Sequence
  Description of Artificial Sequence Synthetic polypeptide
MHHHHHHGGSGGVQVETISPGDGRTFPKRGQTVVVHYTGMLEDGKKVDSSRDRNKPFKFMLGKQEVIRGWEEGVAQMSVGQRAKLTISPDYAYGATGHPGIIPPHATLVFDVELLKLESGGGGSGGGGSGGGSVTGYRLFEEIL